(19) United States
(12) Patent Application Publication
(10) Pub. No.: US 2015/0306170 A1
(43) Pub. Date: Oct. 29, 2015

(54) COMPOSITION FOR IMMEDIATE AND EXTENDED RELEASE

(71) Applicant: FERRING B.V., Hoofddorp (NL)

(72) Inventors: Varinder Ahuja, Mumbal-Maharashtra (IN); Rajan Verma, New Delhi (IN); Umesh Vinayakrao Barabde, Maharashtra (IN); Arne Hagsten, Smorum (DK); Kristin Wannerberger, Pully (CH); Rambabu Boorugu, Andhrapradesh (IN); Amol Vilasrao Somwanshi, Ahmednagar (IN)

(21) Appl. No.: 14/443,759

(22) PCT Filed: Nov. 21, 2013
(86) PCT No.: PCT/EP2013/074373

§ 371 Date: May 19, 2015

(30) Foreign Application Priority Data
Nov. 21, 2012 (IN) 3575/DHL/2012
Dec. 20, 2012 (IN) 3947/DHL/2012

(51) Int. Cl. A61K 38/11; A61K 9/00; A61K 9/16; A61K 31/137

(52) U.S. Cl. CPC A61K 38/11; A61K 31/137; A61K 9/006; A61K 9/1676

(57) ABSTRACT

The subject invention relates to fast dissolving pharmaceutical compositions comprising an active ingredient for immediate release and further comprising a controlled release dosage form comprising an active ingredient for controlled release.

### FIELD OF THE INVENTION

The subject invention relates to fast dissolving pharmaceutical compositions comprising an active ingredient for immediate release and further comprising a controlled release dosage form comprising an active ingredient for controlled release, to methods of making them and to their use in the treatment and prophylaxis of diseases in mammals, particularly humans.

### BACKGROUND

Fast dissolving pharmaceutical dosage forms which are designed to immediately release an active ingredient in the oral cavity are well known and can be used to deliver a wide range of drugs (Critical Reviews in Therapeutic Drug Carrier Systems, 21(6):433-475 (2004); Seager H. (1998), J. Phar. Pharmacol 50:375-382; Bandari et al. (January 2008), Asian Journal of Pharmaceutics 2-11).

In a fast dissolving dosage form, a drug is physically trapped in a matrix composed of a carrier material, e.g., mannitol and fish gelatin (EP 1 501 534; EP 1 165 053), modified starch (U.S. Pat. No. 6,509,040), pullulan in combination with an amino acid (EP 1 803 446), maltodextrin in combination with sorbitol (US 2004/0228919), levan (WO 2011/120904) or inulin (WO 2011/120903). For preparing the fast dissolving dosage form, a solution, suspension or dispersion of the drug and the carrier material may be filled into blister cavities, frozen and thereafter lyophilized.

Controlled release pellets comprising an active ingredient to be released in a controlled fashion are also well known in the art and are for example described in U.S. Pat. No. 6,911,217, US 2009/0192228, EP 1 781 275, and WO 2007/029087. These controlled release beads have typically been used in the pharmaceutical industry within pharmaceutical (hard-gelatin) capsules for oral administration and can be prepared by a wide variety of methods such as layering, extrusion spheronization, granulation, hot melt extrusion, spray drying and the like.

Certain diseases and disorders require one drug to be administered in such a manner as to result in immediate release and another drug to be administered in such a manner as to result in extended release. It would of course be advantageous if these two drugs could be administered in one single dosage unit which would release one drug in an immediate fashion and the other in an extended fashion.

### SUMMARY OF THE INVENTION

The subject invention now provides a formulation not only comprising a first drug for immediate release trapped in a matrix, but additionally comprising another dosage form trapped within the matrix, the other dosage form being extended release beads comprising a second drug for extended release.

The subject invention thus provides a single dose unit pharmaceutical combination product comprising a lyophilized, melt-type, fast dissolving or disintegrating formulation comprising a first active ingredient for immediate release (IR) wherein the fast dissolving formulation further comprises extended release (ER) pellets comprising a second pharmaceutically active ingredient to be released in a controlled fashion. The extended release pellets are physically trapped or embedded in the matrix of the fast dissolving formulation.

The fast dissolving oral pharmaceutical compositions are typically oral lyophilizates (also named orally disintegrating tablets or orally dissolving tablets), comprising a first active ingredient for immediate release (IR) and further comprising extended release (ER) pellets comprising a second pharmaceutically active ingredient to be released in a controlled fashion.

The extended release pellets can be formulated by methods known in the art such as layering, extrusion spheronization, granulation, hot melt extrusion, spray drying and the like.

It has been found that the composition of the present invention, comprising pellets within a fast dissolving formulation, allows to accomplish many unexpected and beneficial technical effects:
Stable release profiles of the first and second drugs (active ingredients), i.e. release profiles which are similar to, substantially identical or identical with those observed in separate compositions comprising only the first drug in an IR formulation and the second drug in an ER formulation, respectively;
A relatively high tensile strength (i.e. force required to break a tablet in a three-point bending test) as compared to a composition without pellets;
A total weight of the composition which is pharmaceutically acceptable and acceptable from a consumer point of view;
A fast disintegration/dissolution time of less than 30 seconds; and
The composition may be prepared by lyophilization with only little, substantially no, or no influence on the extended release profile of the pellet.

The relatively high tensile strength permits, amongst others, to easily remove the composition from its container, typically a blister pack, without disintegration and without risk of damaging the dosage form between the fingers. The unit dosage form of the invention can typically be handled in a manner similar to that of a conventional compressed tablet, with disintegration occurring only upon contact with an aqueous liquid or with saliva within the mouth.

Notwithstanding this tensile strength, the composition of the invention disintegrates rapidly when contacted with an aqueous medium or with saliva, in particular the composition rapidly disintegrates when taken orally.

The pharmaceutical composition of the invention may be obtained by sublimating a solvent (e.g. water), for example in a freeze drying process, from a liquid preparation that comprises the first active ingredient, the matrix-forming agent(s) and the controlled release beads (which again comprise an additional second active ingredient) in solution. According to one embodiment, unit dosage quantities of the liquid preparation are introduced into depressions and sublimation is then carried out, thus obtaining (after sublimation) a pharmaceutical composition in a unit dosage form comprising two active ingredients, one for immediate release and one for controlled release. The depressions may be those of an open blister pack, and following the sublimation step (and thus following the formation of the solid unit dosage form of the composition in the depression), a sealing film or foil is placed over the depressions to form a sealed blister pack.

In particular, the present invention relates to a pharmaceutical composition comprising an open matrix network comprising a first pharmaceutically active ingredient; one or more matrix-forming agents; and controlled release beads comprising a second pharmaceutically active ingredient.

The invention furthermore relates to a process for preparing a pharmaceutical composition comprising sublimating a solvent from a liquid preparation comprising a first pharmaceutically active ingredient, one or more matrix-forming agents, controlled released pellets comprising a second pharmaceutically active ingredient, and a solvent.

The invention also relates to a process for the preparation of a pharmaceutical composition comprising the steps of:
(a) preparing a mixture comprising a first active ingredient, controlled release beads comprising a second active ingredient, one or more matrix-forming agents, and a solvent;
(b) freezing said solution;
(c) sublimating the solvent from the frozen solution,
   wherein the pharmaceutical composition so obtained disintegrates within 30 seconds upon contact with a standardized aqueous medium.

The invention concerns a method for treating overactive bladder, nocturia or a combination thereof in a subject in need thereof, which comprises administering to the subject a therapeutically effective amount of a composition wherein the second active ingredient is an antimuscarinic compound.

The invention concerns a method for treating benign prostatic hyperplasia in a subject in need thereof, which comprises administering to the subject a therapeutically effective amount of a composition wherein the second active ingredient is a selective alpha blocker.

### BRIEF DESCRIPTION OF THE DRAWINGS

FIG. 1a is a schematic representation of a single dose unit pharmaceutical combination product of the invention comprising a lyophilized, melt-type, fast dissolving formulation comprising a first active ingredient for immediate release wherein the fast dissolving formulation further comprises extended release pellets comprising a second pharmaceutically active ingredient to be released in a controlled fashion.

FIG. 1b is a schematic representation of an extended release pellet or bead used in the present invention and comprising:
1: a core
2: an optional inner sealcoat layer
3: an inner drug-containing layer
4: an optional outer sealcoat layer
5: an outer membrane layer and
6: an optional additional polymer layer.

FIG. 1c is a schematic representation of an extended release pellet or bead used in the present invention and comprising a core comprising drug, excipients and optionally controlled release polymers. The core can be optionally coated with a controlled release polymer.

FIG. 2 is a diagram comparing the dissolution profiles of Detrusitol® XL and tolterodine ER beads (particles) and lyophilizates according to Example 1 in pH 6.8 phosphate buffer using United States Pharmacopoeia (USP) Apparatus 1 (basket) at 100 rpm.

FIG. 3 is a diagram comparing the release of first active ingredient (desmopressin) and second active ingredient (tolterodine) from the dosage form of Example 2 in pH 6.8 phosphate buffer using USP Apparatus 2 (paddle) at 50 rpm for desmopressin, and USP Apparatus 1 (basket) at 100 rpm for tolterodine.

FIG. 4 is a diagram comparing the release of first active ingredient (desmopressin) and second active ingredient (tolterodine) from the dosage form of Example 3 in pH 6.8 phosphate buffer using USP Apparatus 2 (paddle) at 50 rpm for desmopressin, and USP Apparatus 1 (basket) at 100 rpm for tolterodine.

FIG. 5 is a diagram comparing the release of first active ingredient (desmopressin) and second active ingredient (tolterodine) from the dosage form of Example 4 in pH 6.8 phosphate buffer using USP Apparatus 2 (paddle) at 50 rpm for desmopressin, and USP Apparatus 1 (basket) at 100 rpm for tolterodine.

FIG. 6 is a diagram comparing the release of first active ingredient (desmopressin) and second active ingredient (tolterodine) from the dosage form of Example 5 in pH 6.8 phosphate buffer using USP Apparatus 2 (paddle) at 50 rpm for desmopressin, and USP Apparatus 1 (basket) at 100 rpm for tolterodine.

FIG. 7 is a diagram comparing the dissolution profiles of Detrusitol® XL and tolterodine ER beads (particles) and lyophilizates according to Example 6 in pH 6.8 phosphate buffer using United States Pharmacopoeia (USP) Apparatus 1 (basket) at 100 rpm.

FIG. 8 is a diagram comparing the dissolution profiles of Detrusitol® XL and tolterodine ER beads (particles) and lyophilizates according to Example 7 in pH 6.8 phosphate buffer using United States Pharmacopoeia (USP) Apparatus 1 (basket) at 100 rpm.

FIG. 9 is a diagram comparing the dissolution profiles of Detrusitol® XL and tolterodine ER beads (particles) and lyophilizates according to Example 8 in pH 6.8 phosphate buffer using United States Pharmacopoeia (USP) Apparatus 1 (basket) at 100 rpm.

FIG. 10 is a diagram comparing the release of first active ingredient (desmopressin) and second active ingredient (tolterodine) from the dosage form of Example 9 in pH 6.8 phosphate buffer using USP Apparatus 2 (paddle) at 50 rpm for desmopressin, and USP Apparatus 1 (basket) at 100 rpm for tolterodine.

FIG. 11 is a diagram comparing the release of first active ingredient (desmopressin) and second active ingredient (tolterodine) from the dosage form of Example 10 in pH 6.8 phosphate buffer using USP Apparatus 2 (paddle) at 50 rpm for desmopressin, and USP Apparatus 1 (basket) at 100 rpm for tolterodine.

FIG. 12 is a diagram comparing the release of first active ingredient (desmopressin) and second active ingredient (tolterodine) from the dosage form of Example 11 in pH 6.8 phosphate buffer using USP Apparatus 2 (paddle) at 50 rpm for desmopressin, and USP Apparatus 1 (basket) at 100 rpm for tolterodine.

FIG. 13 is a diagram comparing the release of first active ingredient (desmopressin) and second active ingredient (tolterodine) from the dosage form of Example 12 in pH 6.8 phosphate buffer using USP Apparatus 2 (paddle) at 50 rpm for desmopressin, and USP Apparatus 1 (basket) at 100 rpm for tolterodine.

FIG. 14 is a diagram comparing the release of first active ingredient (desmopressin) and second active ingredient (tolterodine) from the dosage form of Example 13 in pH 6.8 phosphate buffer using USP Apparatus 2 (paddle) at 50 rpm for desmopressin, and USP Apparatus 1 (basket) at 100 rpm for tolterodine.

FIG. 15 is a diagram comparing the release of first active ingredient (desmopressin) and second active ingredient (tolterodine) from the dosage form of Example 14 in pH 6.8 phosphate buffer using USP Apparatus 2 (paddle) at 50 rpm for desmopressin, and USP Apparatus 1 (basket) at 100 rpm for tolterodine.

### DETAILED DESCRIPTION OF THE INVENTION

It is an object of the invention to provide a dosage form comprising two active ingredients, one of which is to be released in an immediate fashion and one in a controlled fashion.

The subject invention now provides a new orodispersible dosage form comprising two drugs (active ingredients). One of the drugs is released in an immediate fashion, the other one in a controlled fashion. The dosage form is a fast-dissolving dosage form, such as, but not limited to a melt-like or lyophilized unit. The drug released in a controlled fashion is comprised within pellets (beads) which are in turn contained within the fast dissolving formulation. Despite the presence of the pellets within the fast dissolving dosage form, the composition is stable, the weight of the entire composition is pharmaceutically acceptable and acceptable from a consumer point of view, and the oral dissolution time of the fast dissolving dosage form remains fast.

The terms “drug”, “active ingredient” or “pharmaceutically active ingredient” will be used interchangeably herein.

The term “pharmaceutical composition” and “composition” are interchangeably used herein to refer to a pharmaceutical composition of the invention.

The terms “controlled release pellets” or “pellets” or “controlled release beads” or “beads” or “controlled release particles” or “particles” will be used interchangeably herein.

Controlled release beads can be manufactured by several methods known in the art such as layering, extrusion spheronization, granulation, hot melt extrusion, spray drying and so forth wherein the second active ingredient is mixed and/or coated with release controlling agents.

When the controlled release beads are made by layering, the controlled release beads typically contain a pharmaceutically inactive core 1 selected from a water-soluble core, a water-insoluble core and a water-swellable core coated with an inner drug-containing layer 3 and an outer membrane layer 5 controlling drug release from the inner layer.

Some controlled release beads also comprise a “sealcoat” 2 made of a polymer selected from substantially water-insoluble polymer and substantially water-soluble polymer, between the inert core 1 and the inner drug-containing layer 3.

Some controlled release beads also comprise a “sealcoat” 4 made of a polymer selected from substantially water-insoluble polymer and substantially water-soluble polymer, between the inner drug-containing layer 3 and the outer membrane layer 5.

The controlled release beads may further contain an additional polymer layer 6 on the outer membrane layer 5.

It is an object of the invention to provide controlled release beads, the drug release profile of which is not significantly influenced by lyophilization, thus enabling the use of these beads within the lyophilized dosage forms of the invention.

The cores are typically made of a water-soluble, water-insoluble or water-swellable material, and may consist of any material that is conventionally used as cores or any other pharmaceutically acceptable water-soluble, water-insoluble or water-swellable material that can be made into beads or pellets. For example, the cores can be spheres of sucrose/starch (Sugar Spheres NF), sucrose crystals, glass or microcrystalline cellulose. In particular, the cores can be water-soluble sugar spheres or water-swellable microcrystalline cellulose cores. The cores can be made, for instance, by extrusion and subsequent drying extrudates of excipients such as microcrystalline cellulose and lactose.

The substantially water-insoluble polymer in the optional sealcoat layers 2 and 4 situated (i) between the core 1 and the inner drug-containing layer 3 and/or (ii) between the inner drug-containing layer 3 and the outer membrane layer 5 (to control water penetration into the core) is generally a “GI insoluble” (GI=gastrointestinal) or “GI partially insoluble” film-forming polymer. Non-limiting examples of such polymers are ethyl cellulose, cellulose acetate, cellulose acetate butyrate, polymethacrylates such as ethyl acrylate/methyl methacrylate copolymer (Eudragit® NE 30 D) and ammonio methacrylate copolymer types A and B (Eudragit® RL 30 D and RS 30 D), silicone elastomers and mixtures of two or more thereof. In one particular embodiment, the substantially water-insoluble polymer of the inner sealcoat layer 2 comprises ethyl cellulose. Occasionally, one or more plasticizers are used together with the polymer. Non-limiting examples of plasticizers include dibutyl sebacate, propylene glycol, triethyl citrate, tributyl citrate, castor oil, acetylated monoglycerides, acetyl triethyl citrate, acetyl butyl citrate, diethyl phthalate, dibutyl phthalate, triacetin, medium-chain triglycerides such as fractionated coconut oil and so forth.

The water-soluble polymer in the optional sealcoat layers 2 and 4 may be selected from hydrophilic polymers such as polyvinylpyrrolidone (PVP), polyalkylene glycol such as polyethylene glycol, gelatine, polyvinyl alcohol, starch and derivatives thereof, cellulose derivatives, such as hydroxypropylmethyl cellulose (HPMC), hydroxypropyl cellulose, carboxymethyl cellulose, methyl cellulose, ethyl cellulose, hydroxyethyl cellulose, carboxyethyl cellulose, carboxymethylhydroxyethyl cellulose, acrylic acid polymers, polymethacrylates and mixtures of two or more thereof.

The inner layer 3 containing the (second) active ingredient may be comprised of the active ingredient (drug) with or without a polymer as a binder. The binder, when used, is usually hydrophilic and may be water-soluble or water-insoluble. Non-limiting examples of polymers to be used in the inner layer containing the active drug are hydrophilic polymers such as polyvinylpyrrolidone (PVP), polyalkylene glycol such as polyethylene glycol, gelatine, polyvinyl alcohol, starch and derivatives thereof, cellulose derivatives, such as hydroxypropylmethyl cellulose (HPMC), hydroxypropyl cellulose, carboxymethyl cellulose, methyl cellulose, ethyl cellulose, hydroxyethyl cellulose, carboxyethyl cellulose, carboxymethylhydroxyethyl cellulose, acrylic acid polymers, polymethacrylates and mixtures of two or more thereof. In one particular embodiment, the inner drug-containing layer 3 comprises hydroxypropylmethyl cellulose as binder. The ratio of drug to hydrophilic polymer in the inner layer is usually in the range of from 1:5 to 10:1 (w/w).

Suitable polymers for use in the outer membrane layer 5 for controlling the drug release may be selected from water-insoluble polymers or polymers with pH-dependent solubility, such as, for example, ethyl cellulose, hydroxypropylmethyl cellulose phthalate, cellulose acetate phthalate, cellulose acetate trimellitate, polymethacrylates, or mixtures thereof, optionally combined with plasticizers, such as those mentioned above. Optionally, the controlled release layer comprises, in addition to the polymers above, another substance(s) with different solubility characteristics, to adjust the permeability and, thus, the release rate of the second drug. Exemplary polymers that may be used as a modifier together with, for example, ethyl cellulose include: HPMC, hydroxyethyl cellulose, hydroxypropyl cellulose, methylcellulose, carboxymethylcellulose, polyethylene glycol, polyvinylpyrrolidone (PVP), polyvinyl alcohol, polymers with pH-dependent solubility, such as cellulose acetate phthalate or ammonio methacrylate copolymer and methacrylic acid copolymer, or mixtures thereof. Additives such as sucrose, lactose, pharmaceutical grade surfactants and mixtures of two or more thereof may also be included in the controlled release layer. In one particular embodiment, the outer membrane layer 5 comprises a combination of hydroxypropylmethyl cellulose (HPMC) and ethyl cellulose.

Suitable polymers for use in the optional additional polymer layer 6 on the outer membrane layer 5 are those which may provide enteric and/or lyoprotective functionality and may be selected from methacrylic acid copolymers, cellulose acetate phthalate, cellulose acetate butyrate, hydroxypropyl methylcellulose phthalate, hydroxypropyl methylcellulose acetate succinate, polyvinyl acetate phthalate, cellulose acetate trimellitate, carboxymethylethylcellulose, shellac, cellulose ethers (e.g., ethyl cellulose, hypromellose, hyprolose), PVP, acrylate polymers (e.g., Eudragit® NE 30 D, Eudragit® RL, Eudragit® RS) and mixtures of two or more thereof. They may be coated in the form of solutions or dispersions on the outer membrane layer 5. The lyoprotective functionality may be optionally required to avoid dissolution of the outer membrane 5 during lyophilization.

The controlled release beads are prepared by:
a) providing a core 1 unit of a substantially water-soluble, water-insoluble or water-swellable material;
b) optionally applying an inner sealcoat layer 2 of a polymer onto said core 1;
c) applying onto the core 1 or onto the inner sealcoat layer 2 an inner layer 3 containing the (second) active ingredient and optionally a polymer binder;
d) optionally applying an outer sealcoat layer 4 of a polymer onto said inner layer 3;
e) applying onto said inner layer 3 or onto the outer sealcoat layer 4 an outer membrane layer 5 effective for controlled release of the active ingredient; and
f) optionally applying onto said outer membrane layer 5 an additional polymer layer 6.

The layering or coating operations are preferably performed by spraying a solution or dispersion of the respective layer material(s) onto the core, for example in a centrifugal coater, coating pan, Granurex® rotor process, or fluid bed coater, preferably in a fluid bed coater.

After coating of the sealcoat and outer layers, the beads may be “cured”, usually in a fluid bed system or in a tray dryer system, for example by heating to a temperature of about 30-80° C. for about 60 minutes.

In one embodiment, the amount of the optional inner sealcoat layer 2 constitutes from about 4% to about 15% (w/w) of the final bead composition.

In one embodiment, the amount of the inner drug-containing layer 3 constitutes from about 5% to about 25% (w/w) of the final bead composition.

In one embodiment, the amount of the optional outer sealcoat layer 4 constitutes from about 1% to about 25% (w/w) of the final bead composition.

In one embodiment, the amount of the outer membrane layer 5 constitutes from about 25% to about 55% (w/w) of the final bead composition.

In one embodiment, the amount of the optional additional polymer 6 layer constitutes from about 10% to about 35% (w/w) of the final bead composition.

When the controlled release pellets are obtained by hot-melt extrusion, the (second) active ingredient and a controlled release excipient are extruded using known hot-melt extrusion equipment. The resultant extrudes are milled and sieved to obtain the desired fraction. The dried particles of desired particle size are optionally coated with a controlled release polymer. Coating of particles can be performed in appropriate coating equipment, e.g., a centrifugal coater, coating pan, Granurex® rotor process, fluid bed coater and the like.

The controlled release excipient for use in the hot-melt extrusion may be selected from ethyl cellulose, hydroxypropylmethyl cellulose phthalate, cellulose acetate phthalate, cellulose acetate trimellitate, polymethacrylates, HPMC, hydroxyethyl cellulose, hydroxypropyl cellulose, methylcellulose, carboxymethylcellulose, polyethylene glycol, polyvinylpyrrolidone (PVP), polyvinyl alcohol, carbomer, poly(lactide-co-glycolide), polyethylene oxide, glyceryl palmitostearate, glyceryl behenate and mixtures of two or more thereof, optionally combined with plasticizers, such as those mentioned above.

When the controlled release beads are obtained by spray drying, the (second) active ingredient and a controlled release polymer are dissolved or dispersed in a medium. This solution or dispersion is spray dried and resultant particles are optionally dried and sifted to get the desired particle size.

The controlled release polymers for use in the spray drying may be selected from polymers such as, for example, ethyl cellulose, hydroxypropylmethyl cellulose phthalate, cellulose acetate phthalate, cellulose acetate trimellitate, polymethacrylates, HPMC, hydroxyethyl cellulose, hydroxypropyl cellulose, methylcellulose, carboxymethylcellulose, polyethylene glycol, polyvinylpyrrolidone (PVP), polyvinyl alcohol and mixtures of two or more thereof, optionally combined with plasticizers, such as those mentioned above.

When the controlled release beads are obtained by granulation, the (second) active ingredient is dry-mixed with excipients and granulated using a binder solution. The granules are dried and sieved to get the desired fraction. The dried granules of desired particle size can be further coated with a controlled release polymer. Alternatively, the active ingredient and a controlled release excipient is dry-mixed and granulated using a binder solution. The granules are then dried and sieved to get the desired fraction. The dried granules of desired particle size can optionally be further coated with a controlled release polymer.

The process of granulation is performed in a granulator such as, but not limited to, a rapid mixer granulator, planetary mixer, fluid bed processer, centrifugal granulator, and the like. Coating of granules may be performed in appropriate coating equipment such as a centrifugal coater, coating pan, Granurex® rotor process, fluid bed coater and the like.

The controlled release polymers for use in the granulation may be selected from polymers such as, for example, ethyl cellulose, hydroxypropylmethyl cellulose phthalate, cellulose acetate phthalate, cellulose acetate trimellitate, polymethacrylates, HPMC, hydroxyethyl cellulose, hydroxypropyl cellulose, methylcellulose, carboxymethylcellulose, polyethylene glycol, polyvinylpyrrolidone (PVP), polyvinyl alcohol and mixtures of two or more thereof, optionally combined with plasticizers, such as those mentioned above.

When the controlled release beads are obtained by extrusion-spheronization, the (second) active ingredient, filler and optionally a controlled release polymer is dry-mixed and granulated in a granulator such as a rapid mixer granulator, planetary mixer, fluid bed processer, centrifugal granulator and the like. The wet mass obtained is then extruded and spheronized to form spherical particles. The spherical particles are dried and sieved to get the desired fraction. The dried particles of desired particle size can optionally be further coated with a controlled release polymer. Alternatively, the extruded particles are dried and sieved to get the desired fraction. The dried particles of desired particle size can optionally be further coated with a controlled polymer. Coating of particles is performed in appropriate coating equipment, e.g., centrifugal coater, coating pan, Granurex® rotor process, fluid bed coater, and the like.

The controlled release polymers for use in the extrusion-spheronization may be selected from polymers such as, for example, ethyl cellulose, hydroxypropylmethyl cellulose phthalate, cellulose acetate phthalate, cellulose acetate trimellitate, polymethacrylates, HPMC, hydroxyethyl cellulose, hydroxypropyl cellulose, methylcellulose, carboxymethylcellulose, polyethylene glycol, polyvinylpyrrolidone (PVP), polyvinyl alcohol and mixtures of two or more thereof, optionally combined with plasticizers, such as those mentioned above.

In one embodiment, the extended release profile of the beads used in the subject invention is resistant to, i.e. not substantially influenced by, the lyophilization process used to prepare the pharmaceutical composition of the invention.

The term “matrix” should be understood to denote a solid carrier medium for an active ingredient. The matrix comprises one or more excipients. The excipients that form the matrix are herein referred to as “matrix-forming agents” and each of said agents as “matrix-forming agent”.

The term “an open matrix network” should be understood to encompass a matrix of water-soluble or water-dispersible carrier material (matrix-forming agent(s)) having interstices dispersed throughout. The matrix rapidly disintegrates upon contact with an aqueous medium or with saliva.

Unless defined otherwise, percentages within the specification and claims are based on weight (wt. % or w/w).

The matrix-forming agent in the composition can be any agent capable of forming a matrix of water-soluble or water-dispersible carrier material. Non-limiting examples of matrix-forming agents are levan, inulin, pullulan, sodium alginate, fish gelatin, beta-limit dextrin (BLD), modified starch, maltodextrin (optionally in combination with sorbitol), acacia, hydroxypropyl methylcellulose and/or pectin and any combinations thereof. In one embodiment, the matrix-forming agents are selected from the group consisting of levan, inulin, pullulan, acacia, maltodextrin, HPMC, sodium alginate and combinations thereof.

Levan (also named leaven, levulosan, polyfructosan, polyfructose and polylevulan) is a polymer of fructose, C₆H₁₂O₆. Levan is a polysaccharide with β-(2->6) linkages between the fructose rings where the numbers describe the carbon atoms in the fructose ring which are linked and the β describes the stereochemical relationship. Levans have also been described as fructans in which the predominant glycosidic linkage between the D-fructofuranoside monomeric units is β-(2->6). The levans are generally made by microorganisms and do not occur as high molecular weight compounds in plants. Some low molecular weight levans having a molecular weight of less than 100,000 Daltons can occur in grasses.

“Levan” as used herein should be understood to encompass levan derived from any source such as but not limited to Aspergillus indicus, Aspergilllus versicolor, Acetobacter suboxydans, Achromobacter spp., Actinomycenes sp., Actinomyces viscosus, Aerobacter aerogenes, Aerobacter levanicum, Aspergillus sydowii, Azotobacter chroococcum, Bacillus polymyxa, Bacillus licheniformis, Bacillus macerans, Bacillus megatherium, Bacillus mesentericus, Bacillus subtilis, Bacillus vulgatus, Corynbacterium laevaniformans, Erwinia herbicola, Gluconobacter oxydans, Leuconostoc mesenteroides, Odontomyces viscosus, Phytobacterium vitrosum, Phytomonas pruni, Psuedomonas Fluorescens, Pseudomonas Syringae, Pseudomonas prunicola, Rothis dentocariosa, Serratia kiliensis, Steptococcus bovis, Steptococcus mutans, Steptococcus salivarius, Xanthomonas campestris, Xanthomonas pruni, Zymomonas mobilis and so forth. In a specific embodiment, the levan is obtained from Zymomonas and Bacillus species. In a more specific embodiment, the levan is obtained from Zymomonas mobilis.

It should be understood that also derivatives of levan (e.g. as described in WO 98/03184) can be used in place of levan.

Inulin is a polymer of fructose, C₆H₁₂O₆, typically having a terminal glucose. Inulin is a polysaccharide with β-(2->1) linkages between the fructose rings where the numbers describe the carbon atoms in the fructose ring which are linked and the β describes the stereochemical relationship. The inulins are produced by many types of plants.

Inulin as used herein should be understood to encompass inulin derived from any source such as but not limited to plants that contain high concentrations of inulin which include, but are not limited to Elecampane (Inula helenium); Dandelion (Taraxacum officinale); Wild Yam (Dioscorea spp.); Jerusalem artichokes (Helianthus tuberosus); Chicory (Cichorium intybus); Jicama (Pachyrhizus erosus); Burdock (Arctium lappa); Onion (Allium cepa); Garlic (Allium sativum); Agave (Agave spp.); Yacón (Smallanthus sonchifolius spp.); and Camas (Camassia spp.). In a specific embodiment, the inulin is obtained from Chicory (Cichorium intybus).

One or more secondary matrix-forming agents may be present in the composition. Non-limiting examples of sugars, sugar alcohols, monosaccharides, disaccharides, trisaccharides, polysaccharides, proteins, amino acids, gums and the like, which are useful as secondary matrix-forming agents, include without limitation, mannitol, trehalose, raffinose, inositol, pullulan, sucrose, lactose, dextrose, erythritol, xylitol, lactitol, maltitol, isomalt, alanine, arginine, threonine, glycine, cysteine, serine, histidine, valine, proline, lysine, asparagine, glutamine, ribose, glucose, galactose, fructose, maltose, maltotriose, guar gum, xanthan gum, tragacanth gum, veegum, microcrystalline cellulose, sodium carboxymethyl cellulose and so forth.

Generally, the balance of the formulation can be matrix. Thus the percentage of the matrix can approach 100%. The amount of a secondary matrix-forming agent useful in accordance with the present invention may range from about 0 to about 30%.

In one embodiment of the invention, levan is the main matrix-forming agent in the composition. In another embodiment, inulin is the main matrix-forming agent. In yet another embodiment, both levan and inulin are used in combination as the main matrix forming agents.

In another embodiment, the composition further comprises mannitol or raffinose or trehalose or combinations thereof as secondary matrix-forming agent(s) in the open matrix network.

In one embodiment, levan is the matrix-forming agent, constituting 10-50% of the entire weight of the composition. In another embodiment, levan constitutes 20-40% of the entire weight of the composition. In yet another embodiment, levan constitutes 25-35% of the entire weight of the composition.

In other embodiments, mannitol or trehalose or raffinose or combinations thereof are used as secondary matrix-forming agents, constituting 10-40% of the entire weight of the composition. In one embodiment, these secondary matrix-forming agents constitute 20-30% of the entire weight of the composition.

Thus, a composition of the invention can be one comprising levan as the main matrix-forming agent and mannitol or trehalose or raffinose (or combinations thereof) as secondary matrix-forming agent, with levan constituting 10-50% (all % of ingredient are w/w, meaning weight of mentioned ingredient out of the weight of all constituents of the composition combined), and the secondary matrix-forming agent constituting 10-40%, typically 20-30%.

The content of the first active ingredient may typically (but not exclusively) be in the range of 0.01-1% of the entire composition, typically in the range of 0.02-0.2% depending on the nature of the active ingredient. The content of the second active ingredient may typically (but not exclusively) be in the range of 1-50% of the entire composition, typically in the range of 3-10% depending on the nature of the active ingredient. In one embodiment, the active ingredients constitute about 4% of the entire weight of the composition. In another embodiment, the active ingredients constitute about 5% of the entire weight of the composition. In yet another embodiment, the active ingredients constitute about 6% of the entire weight of the composition. In other embodiments, the active ingredients constitute 7% of the entire weight of the composition. In yet other embodiments, the active ingredients constitute 8% of the entire weight of the composition.

The term “disintegration” refers to the dissolution or “falling apart” of the matrix with the first drug for immediate release, and setting free or releasing the extended release dosage form, such as pellets or beads, thus allowing them to start releasing the second drug, depending on the conditions in the aqueous medium. The disintegration in a standardized aqueous medium is typically within less than 30 seconds, and more typically within less than 10 seconds, or even less than 9, 8, 7, 6, 5, 4, 3, 2 or even 1 second.

“Disintegration time” and “Dissolution time” are used interchangeably herein and should be understood to mean the time needed to dissolve or disintegrate the composition of the invention in a standardized aqueous medium.

“Oral dissolution time” as used herein should be understood to mean the time needed to dissolve the composition of the invention in the oral cavity.

“Rapid/fast disintegration/dissolution” as used herein should be understood to encompass disintegration/dissolution of the composition of the invention in a standardized aqueous medium within 30 seconds, typically within 20, preferably within 10 seconds, or even within 9, 8, 7, 6, 5, 4, 3, 2 or 1 second.

Examples of an aqueous medium as used herein are water or a buffer (e.g. potassium dihydrogen phosphate, dipotassium hydrogen phosphate, sodium hydrogen phosphate) or artificial saliva as described by Morjaria et al. (May 2004), Dissolution Technologies 12-15. A “standardized aqueous medium” as used herein for the determination of the disintegration time is as defined in the experimental section. The method for determination of the disintegration time is as described in the experimental section.

Saliva as used herein refers to the saliva in the oral cavity of a mammal, in particular a human.

“Tensile strength” as used herein should be understood to be the force required to break a tablet, which is measured by the three-point bending test, wherein the tablet is subjected to a bending stress (Mohd et al. (2002), Drug Development and Industrial Pharmacy 28(7):809-813).

In one embodiment, a pharmaceutical composition of the invention has a tensile strength in the range of about 0.05 to 2 N/mm². In another embodiment, a pharmaceutical composition of the invention has a tensile strength in the range of about 0.05 to 0.3 N/mm². In another embodiment, a pharmaceutical composition of the invention has a tensile strength in the range of about 0.1-0.25 N/mm². In yet another embodiment, a pharmaceutical composition of the invention has a tensile strength in the range of about 0.11-0.23 N/mm².

It is envisaged that a pharmaceutical composition of the invention has a rapid disintegration/dissolution rate such that the composition is dissolved in a standardized aqueous medium within 30 seconds, typically within 10 seconds.

In one embodiment, a pharmaceutical composition of the invention has a tensile strength in the range of about 0.05-2 N/mm² and a rapid disintegration/dissolution rate such that the composition is dissolved in a standardized aqueous medium within 30 seconds, typically within 10 seconds.

In another embodiment, a pharmaceutical composition of the invention has a tensile strength in the range of about 0.05-0.3 N/mm² and a rapid disintegration/dissolution rate such that the composition is dissolved in a standardized aqueous medium within 30 seconds, typically within 10 seconds.

In another embodiment, the invention provides a pharmaceutical composition comprising first and second pharmaceutically active ingredients, having a tensile strength ranging between about 0.05-2 N/mm² and a rapid disintegration/dissolution rate such that the composition is dissolved in a standardized aqueous medium within 30 seconds, typically within 10 seconds.

In another embodiment, the invention provides a pharmaceutical composition comprising first and second pharmaceutically active ingredients, having a tensile strength ranging between about 0.05-0.3 N/mm² and a rapid disintegration/dissolution rate such that the composition is dissolved in a standardized aqueous medium within 30 seconds, typically within 10 seconds.

The open matrix network enables a liquid to enter the dosage form through the interstices and permeate through its interior. Permeation by aqueous media (such as saliva, water, etc.) exposes the carrier material of both the interior and exterior of the dosage form to the action of the aqueous media or saliva whereby the network of carrier material is rapidly disintegrated/dissolved thereby releasing the first active ingredient and the controlled release pellets into the oral cavity.

The open matrix structure is of a porous nature and enhances disintegration of the dosage form as compared with ordinary solid shaped pharmaceutical dosage forms such as (granulated and compressed) tablets, pills, capsules, suppositories and pessaries. Rapid disintegration results in rapid release of the active ingredient comprised in the matrix and further results in release of the controlled release beads which are swallowed/absorbed and which will release their active ingredient in a controlled fashion.

The pharmaceutically active ingredients used in the subject invention can be any pharmaceutically active ingredient such as a low molecular weight compound, a peptide, a nucleotide, and so forth.

The first and second active ingredients may be identical or different from each other. In one embodiment, they are different from each other.

Non-limiting examples of drugs (active ingredients) which can be comprised in the open matrix network of the subject invention and/or comprised within the pellets comprised within the open matrix network are analgesics, alpha blockers, anti-allergy, anti-asthma, (allergic rhinitis, chronic uticaria), anti-inflammatory, antacids, anthelmintics, anti-arrhythmic agents, anti-arthritis, anti-bacterial, anti-anxiety, anti-coagulants, anti-depressants, anti-diabetics, anti-diarrheals, anti-diuretics, anti-epileptics, anti-fungal, anti-gout, anti-hypertensive, anti-incontinence, anti-insomnia, anti-malarials, anti-migraine, anti-muscarinic, anti-neoplastic and immunosuppressants, anti-protozoal, anti-rheumatics, anti-rhinitis, anti-spasmatic. anti-thyroid, antivirals, anxiolytics, sedatives, hypnotics and neuroleptics, beta-blockers, anti-benign hyperplasia (BHP), cardiac inotropic, corticosteroids, cough suppressants, cytotoxics, decongestants, diabetic gastric stasis, diuretics, enzymes, anti-parkinsonian, gastro-intestinal, histamine receptor antagonists, infertility, endometriosis, hormone replacement therapy, lipid regulating agents, local anesthetics, neuromuscular agents, nitrates and anti-anginal agents, menstrual disorders, motion sickness, anti-pain, anti-nausea, movement disorders, nutritional agents, opioid analgesics, oral vaccines, proteins, peptides and recombinant drugs, prevention of chemotherapy induced and post operative nausea and vomiting proton pump inhibitors, schizophrenia, sex hormones and contraceptives, seizure/panic disorder, sexual dysfunction (male and female), spermicides, stimulants voiding dysfunctions, veterinary medicines and so forth.

Specific non-limiting examples of these drugs are:
Alfa blockers: Tamsulosine
Analgesics and anti-inflammatory agents: aspirin, aloxiprin, auranofin, azapropazone, benorylate, diflunisal, etodolac, fenbufen, fenoprofen calcium, flurbiprofen, ibuprofen, indomethacin, ketoprofen, meclofenamic acid, mefenamic acid, nabumetone, naproxen, oxaprozin, oxyphenbutazone, phenylbutazone, piroxicam, sulindac, paracetamol.
Antacids: aluminum hydroxide, magnesium carbonate, magnesium trisilicate, hydrotalcite, dimethicone.
Antihelmintics: albendazole, bephenium hydroxynaphthoate, cambendazole, dichlorophen, ivermectin, mebendazole, oxamniquine, oxfendazole, oxantel embonate, praziquantel, pyrantel embonate, thiabendazole.
Anti-allergic: des loratidine, loratidine, Montelukast, Montelukast sodium, Cetirizin, Fexofenadin, Ebastine.
Anti-arrhythmic agents: amiodarone HCl, disopyramide, flecainide acetate, quinidine sulphate.
Anti-bacterial agents: benethamine penicillin, cinoxacin, ciprofloxacin HCl, clarithromycin, clofazimine, cloxacillin, demeclocycline, doxycycline, erythromycin, ethionamide, imipenem, nalidixic acid, nitrofurantoin, rifampicin, spiramycin, sulphabenzamide, sulphadoxine, sulphamerazine, sulphacetamide, sulphadiazine, sulphafurazole, sulphamethoxazole, sulphapyridine, tetracycline, trimethoprim.
Anti-coagulants: dicoumarol, dipyridamole, nicoumalone, phenindione.
Anti-depressants: amoxapine, ciclazindol, maprotiline HCl, mianserin HCl, nortriptyline HCl, trazodone HCl, trimipramine maleate.
Anti-diabetics: acetohexamide, chlorpropamide, glibenclamide, gliclazide, glipizide, tolazamide, tolbutamide.
Anti-diarrheals: atropine sulphate, codeine phosphate, co-phenotrope, difenoxin, loperamide hydrochloride, suphasolazine, mesalazine, olsalazine, corticosteroids, prednisolone.
Anti-diuretics: desmopressin, desmopressin acetate.
Anti-epileptics: beclamide, carbamazepine, clonazepam, ethotoin, methoin, methsuximide, methylphenobarbitone, oxcarbazepine, paramethadione, phenacemide, phenobarbitone, phenytoin, phensuximide, primidone, sulthiame, valproic acid.
Anti-fungal agents: amphotericin, butoconazole nitrate, clotrimazole, econazole nitrate, fluconazole, flucytosine, griseofulvin, itraconazole, ketoconazole, miconazole, natamycin, nystatin, sulconazole nitrate, terbinafine HCl, terconazole, tioconazole, undecenoic acid.
Anti-gout agents: allopurinol, probenecid, sulphinpyrazone.
Anti-hypertensive agents: amlopidine, benidipine, darodipine, dilitazem HCl, diazoxide, felodipine, guanabenz acetate, indoramin, isradipine, minoxidil, nicardipine HCl, nifedipine, nimodipine, phenoxybenzamine HCl, prazosin HCl, reserpine, terazosin HCl.
Anti-insomnia: Zolpidem Anti-malaria: amodiaquine, chloroquine, chloroproguanil HCl, halofantrine HCl, mefloquine HCl, proguanil HCl, pyrimethamine, quinine sulphate.
Anti-migraine agents: rizatriptan, dihydroergotamine mesylate, ergotamine tartrate, methysergide maleate, pizotifen maleate, sumatriptan succinate, caffeine.
Anti-muscarinic agents: tolterodine, tolterodine tartrate, oxybutinin, atropine, benzhexol HCl, biperiden, ethopropazine HCl, hyoscine butyl bromide, hyoscyamine, mepenzolate bromide, orphenadrine, oxyphencylcimine HCl, tropicamide.
Anti-neoplastic agents and Immunosuppressants: aminoglutethimide, amsacrine, azathioprene, busulphan, chlorambucil, cyclosporin, dacarbazine, estramustine, etoposide, lomustine, melphalan, mercaptopurine, methotrexate, mitomycin, mitotane, mitozantrone, procarbazine HCl, tamoxifen citrate, testolactone.
Anti-protozoal agents: benznidazole, clioquinol, decoquinate, diiodohydroxyquinoline, diloxanide furcate, dinitolmide, furzolidone, metronidazole, nimorazole, nitrofurazone, ornidazole, tinidazole.
Anti-rheumatics: ibuprofen, aceclofenac, acemetacin, azapropazone, diclofenac sodium, diflunisal, etodolac, ketoprofen, indomethacin, mefenamic acid, naproxen, piroxicam, aspirin, benorylate, auranofin, penicillamine.
Anti-rhinitis, anti-uticaria: Cetirizin, fexofenadin, ebastine, loratidin, montelukast
Anti-spasmatic: phloroglucinol anhydre
Anti-thyroid agents: carbimazole, propylthiouracil.
Antivirals: acyclovir, amantadine hydrochloride, famciclovir, zidovadine, didanosine, zalcitabine, foscarnet sodium.
Anxiolytic, sedatives, hypnotics and neuroleptics: alprazolam, amylobarbitone, barbitone, bentazepam, bromazepam, bromperidol, brotizolam, butobarbitone, carbromal, chlordiazepoxide, Chlorpheniramine, chlormethiazole, chlorpromazine, clobazam, clonazepan, clotiazepam, clozapine, diazepam, droperidol, ethinamate, flunanisone, flunitrazepam, fluopromazine, flupenthixol decanoate, fluphenazine decanoate, flurazepam, haloperidol, lorazepam, lormetazepam, medazepam, meprobamate, methaqualone, midazolam, nitrazepam, oxazepam, pentobarbitone, perphenazine phenylephrine, pimozide, prochlorperazine, pseudoephedrineHCL, sulpride, temazepam, thioridazine, triazolam, zopiclone.
β-Blockers: acebutolol, alprenolol, atenolol, labetalol, metoprolol, nadolol, oxprenolol, pindolol, propanolol.
Cardiac inotropic agents: amrinone, digitoxin, digoxin, enoximone, lanatoside C, medigoxin.
Corticosteroids: beclomethasone, betamethasone, budesonide, cortisone acetate, desoxymethasone, dexamethasone, fludrocortisone acetate, flunisolide, flucortolone, fluticasone propionate, hydrocortisone, methylprednisolone, prednisolone, prednisone, triamcinolone.
Cough suppressants: codeine phosphate dexomethorphan, guaifenesin, pholcodine, diamorphine, methadone.
Cytotoxics: ifosfamide, chlorambucil, melphalan, busulphan, cytotoxic antibodies, doxorubicin, epirubicin, plicamycin, bleomycin, methotrexate, cytarabine, fludarabine, gencitabine, fluorouracil, mercaptopurine, thioguanine, vincristine, vinblastine, vindesine, etoposide.
Decongestants: pseudoephedrine hydrochloride.
Diuretics: acetazolamide, amiloride, bendrofluazide, bumetanide, chlorothiazide, chlorthalidone, ethacrynic acid, frusemide, metolazone, spironolactone, triamterene.
Enzymes: pancreatin, pepsin, lipase.
Epilepsy: Gabapentin
Anti-parkinsonian agents: bromocriptine mesylate, lysuride maleate, selegiline, para-fluoroselegiline, lazabemide, rasagiline, 2-BUMP [N-(2-butyl)-N-methylpropargylamine], M-2-PP [N-methyl-N-(2-pentyl)-propargylamine], MDL-72145 [beta-(fluoromethylene)-3,4-dimethoxy-benzeneethanamine], mofegiline, apomorphine, N-propylnoraporphine, cabergoline, metergoline, naxagolide, pergolide, piribedil, ropinirole, terguride, quinagolide.
Gastro-intestinal agents: bisacodyl, cimetidine, cisapride, diphenoxylate HCl, domperidone, metoclopramide, famotidine, loperamide, mesalazine, nizatidine, esomeprazole, metopimazine, pantoprazole, ondansetron HCl, Granisetron, tropisetron, dolasetron, ranitidine HCl, sulphasalazine. Lanzoprazole,
Histamine Receptor Antagonists: acrivastine, astemizole, cinnarizine, cyclizine, cyproheptadine HCl, dimenhydrinate, flunarizine HCl, loratadine, meclozine HCl, oxatomide, terfenadine, triprolidine.
Hormone replacement therapy: dydrogesterone
Hypertension: Enalapril
Lactation: Oxytocin, oxytocin agonists
Lipid regulating agents: bezafibrate, clofibrate, fenofibrate, gemfibrozil, probucol.
Local anaesthetics: amethocaine, amylocaine, benzocaine, bucricaine, bupivacaine, butacaine, butanilicaine, butoxycaine, butyl aminobenzoate, carticaine, chloroprocaine, cinchocaine, clibucaine, clormecaine, coca, cocaine, cyclomethycaine, dimethisoquin, diperodon, dyclocaine, ethyl chloride, ethyl p-piperidinoacetylaminobenzoate, etidocaine, hexylcaine, isobutamben, ketocaine, lignocaine, mepivacaine, meprylcaine, myrtecaine, octacaine, oxethazaine, oxybuprocaine, parethoxycaine, pramoxine, prilocaine, procaine, propranocaine, propoxycaine, proxymetacaine, ropivacaine, tolycaine, tricaine, trimecaine, vadocaine.
Motion sickness: diphenhydramine
Neuro-muscular agents: pyridostigmine.
Nitrates and other anti-anginal agents: amyl nitrate, glyceryl trinitrate, isosorbide dinitrate, isosorbide mononitrate, pentaerythritol tetranitrate.
Nutritional agents: betacarotene, vitamins, such as vitamin A, vitamin B₂, vitamin D, vitamin E, vitamin K, minerals.
Opioid analgesics: codeine, dextropropyoxyphene, diamorphine, dihydrocodeine, meptazinol, methadone, morphine, nalbuphine, pentazocine.
Oral vaccines: to prevent or reduce the symptoms of diseases such as Influenza, Tuberculosis, Meningitis, Hepatitis, Whooping Cough, Polio, Tetanus, Diphtheria, Malaria, Cholera, Herpes, Typhoid, HIV, AIDS, Measles, Lyme disease, Traveller's Diarrhea, Hepatitis A, B and C, Otitis Media, Dengue Fever, Rabies, Parainfluenza, Rubella, Yellow Fever, Dysentery, Legionnaires Disease, Toxoplasmosis, Q-Fever, Haemorrhegic Fever, Argentina Haemorrhegic Fever, Caries, Chagas Disease, Urinary Tract Infection caused by E. coli, Pneumococcal Disease, Mumps, Chikungunya, Hayfever, Asthma, Rheumatoid Arthritis, Carcinomas, Coccidiosis, Newcastle Disease, Enzootic pneumonia, Feline leukemia, Atrophic rhinitis, Erysipelas, Foot and Mouth disease and Swine pneumonia, or to prevent or reduce the symptoms of diseases caused by Vibrio species, Salmonella species, Bordetella species, Haemophilus species, Toxoplasmosis gondii, Cytomegalovirus, Chlamydia species, Streptococcal species, Norwalk Virus, Escherischia coli, Helicobacter pylori, Rotavirus, Neisseria gonorrhae, Neisseria meningiditis, Adenovirus, Epstein Barr Virus, Japanese Encephalitis Virus, Pneumocystis carini, Herpes simplex, Clostridia species, Respiratory Syncytial Virus, Klebsiella species, Shigella species, Pseudomonas aeruginosa, Parvovirus, Campylobacter species, Rickettsia species, Varicella zoster, Yersinia species, Ross River Virus, J.C. Virus, Rhodococcus equi, Moraxella catarrhalis, Borrelia burgdorferi and Pasteurella haemolytica.
Voiding dysfunctions: Tamsulosine, trospium chloride, tolterodine, oxybutinin
Proteins, peptides and recombinant drugs: recombinant hormones and iso-hormones, recombinant cytokines, recombinant plasminogens, TNF receptor fusion protein, monoclonal antibodies, nucleic acids, antisense oligonucleotides, oligonucleotides, glycoproteins and adhesion molecules.
Veterinary Arthiritis: Tepoxalin
Sex hormones and Contraceptives: clomiphene citrate, danazol, desogestrel, ethinyloestradiol, ethynodiol, ethynodiol diacetate, levonorgestrel, medroxyprogesterone acetate, mestranol, methyltestosterone, norethisterone, norethisterone enanthate, norgestrel, estradiol, conjugated estrogens, dydrogesterone, progesterone, stanozolol, stilboestrol, testosterone, tibolone.
Schizoprenia; Olanzapine, Nicergoline
Sexual dysfunction: Cabergolin, oxytocin, tadalafil, sildenafil, vardenafil
Spermicides: nonoxynol 9.
Stimulants: amphetamine, dexamphetamine, dexfenfluramine, fenfluramine, mazindol, pemoline.

In one embodiment, the first active ingredient is desmopressin or a pharmaceutically acceptable salt thereof, especially desmopressin acetate.

In one embodiment, the second active ingredient (comprised within the controlled release beads) is an antimuscarinic compound. In another embodiment, the second active ingredient is selected from tolterodine ((R)-N,N-diisopropyl-3-(2-hydroxy-5-methylphenyl)-3-phenylpropanamine), the 5-hydroxymethyl metabolite of tolterodine ((R)-N,N-diisopropyl-3-(2-hydroxy-5-hydroxymethylphenyl)-3-phenylpropanamine), the (S)-enantiomer of tolterodine ((S)-N,N-diisopropyl-3-(2-hydroxy-5-methylphenyl)-3-phenylpropanamine), the 5-hydroxymethyl metabolite of the (S)-enantiomer of tolterodine ((S)-N,N-diisopropyl-3-(2-hydroxy-5-hydroxymethylphenyl)-3-phenylpropanamine), the racemate of tolterodine ((R,S)-N,N-diisopropyl-3-(2-hydroxy-5-methylphenyl)-3-phenylpropanamine), its prodrug forms and pharmacologically acceptable salts thereof. In a specific embodiment, the active ingredient is tolterodine or a pharmacologically acceptable salt thereof. In a particular embodiment, the active ingredient is tolterodine tartrate.

In a specific, non-limiting embodiment, the first active ingredient comprised in the open matrix network is desmopressin or a pharmaceutically acceptable salt thereof, especially desmopressin acetate, and the second active ingredient comprised in the controlled release beads is tolterodine or a pharmaceutically acceptable salt thereof, especially tolterodine tartrate. The combination of desmopressin acetate and tolterodine tartrate as first and second active ingredients, respectively, is particularly preferred. In this embodiment, the composition can be used in the treatment of urinary disorders, such as, but not limited to overactive bladder or overactive bladder with nocturia, in particular in women.

The overactive bladder condition gives rise to urinary frequency, urgency and/or urge incontinence. Overactive bladder disorders may also include nocturia, i.e. awakening at night to urinate. While overactive bladder is often associated with detrusor muscle instability, disorders of bladder function may also be due to neuropathy of the central nervous system (detrusor hyperreflexia) including spinal cord and brain lesions, such as multiple sclerosis and stroke. Overactive bladder symptoms may also result from, for example, male bladder outlet obstruction (usually due to prostatic hypertrophy), interstitial cystitis, local edema and irritation due to focal bladder cancer, radiation cystitis due to radiotherapy to the pelvis, and cystitis.

In one embodiment, the amount of desmopressin acetate in the composition constitutes 0.01-1% w/w and the amount of tolterodine tartrate in the composition constitutes 3 to 10% w/w. In another embodiment, the amount of desmopressin acetate in the composition constitutes 0.02-0.2% w/w and the amount of tolterodine tartrate in the composition constitutes 3 to 6% w/w.

When tolterodine is the active ingredient in the controlled release bead, the fraction of active ingredient that is released in vitro is preferably not more than about 40% after 1 hour, from about 35 to about 85% after 3 hours, and not less than about 65% after 7 hours.

When tolterodine is the active ingredient in the controlled release beads, the release profile of the tolterodine from the beads (despite their presence within the matrix) will be similar or even identical to the release profile of the tolterodine hard capsules commercially available under the brand name DETRUSITOL XL™.

In one embodiment, the second active ingredient (comprised within the controlled release beads) is a selective alpha-blocker. In another embodiment, the second active ingredient is tamsulosin ((R)-5-(2-{[2-(2-ethoxyphenoxyl)ethyl]amino}propyl)-2-methoxybenzene-1-sulfonamide), its prodrug forms and pharmacologically acceptable salts thereof. In a specific embodiment, the second active ingredient is tamsulosin hydrochloride.

In a specific, non-limiting embodiment, the first active ingredient comprised in the open matrix network is desmopressin or a pharmaceutically acceptable salt thereof, especially desmopressin acetate, and the second active ingredient comprised in the controlled release beads is tamsulosin, or a pharmaceutically acceptable salt thereof, especially tamsulosin hydrochloride. The combination of desmopressin acetate and tamsulosin hydrochloride as first and second active ingredients, respectively, is particularly preferred. In these embodiments, the composition can be used in the treatment of benign prostatic hyperplasia (BPH) in men.

A pharmaceutical dosage form of the invention disintegrates, thereby releasing the active ingredient and the controlled release beads, upon contact with a fluid (an aqueous medium or saliva).

Typically, a pharmaceutical dosage form of the invention is an orodispersible pharmaceutical dosage form which disintegrates in the mouth within 30 seconds, typically 20 seconds or less, preferably 15 seconds or less, more preferably 10 seconds or less and even more preferably within 9, 8, 7, 6, 5, 4, 3, 2 or 1 second.

The term “orodispersible” as used herein should be understood to encompass a solid dosage form which disintegrates or dissolves in water within (at most) 30 seconds when measured according to Ph. Eur. 1997, section 2.9.1, in water at 37° C.±0.5° C.

A suitable route of administration for the dosage form of the subject invention is oral administration, including buccal and sublingual administration. In a specific embodiment, the dosage form is administered sublingually. Dosage forms of the invention may also be placed on the tongue, under the tongue or against the cheek or gingiva.

Pharmaceutical dosage forms of the present invention are adapted to supply the first active ingredient and the controlled release beads to e.g. the oral cavity. The first active ingredient may be absorbed across the mucosa at the site of administration, e.g. sublingual mucosa, and/or otherwise, in the case of oral administration, from the oral cavity (e.g. across the buccal and/or gingival mucosa) and/or from the gastrointestinal tract for systemic distribution.

The exact dose and regimen of administration of the dosage form will necessarily be dependent upon the therapeutic effect to be achieved and may vary with the particular active ingredients, the route of administration, and the age and condition of the individual subject to whom the medicament is to be administered. At times patients may be instructed to take two or any other number of unit dosage forms in a single administration or at times only a portion, such as half or a quarter of the unit dosage form in a single administration.

The dosage form of the invention achieves a balance of performance: tensile strength, stability, uniformity, and fast disintegration. It may be produced by known lyophilizate technology. It can be stored (and packed) in blisters but due to its tensile strength, can also be stored and/or packaged in bottles or bulk. The invention achieves these results in a single processing step, without the need to resort to multiple steps including granulation.

In addition to the ingredients previously discussed, the matrix may also include other excipients (auxiliary agents, accessory agents) such as, but not limited to fillers, thickeners, binders, diluents, lubricants, pH adjusting agents, protecting agents, viscosity enhancers, wicking agents, non-effervescent disintegrants, effervescent disintegrants, surfactants, anti-oxidants, wetting agents, colorants, flavouring agents, taste-masking agents, sweeteners, preservatives and so forth.

In one embodiment, a composition of the invention is obtainable by sublimating solvent from a liquid preparation comprising a first active ingredient, matrix-forming agent(s), controlled release pellets and optionally secondary matrix-forming agent(s) in a solvent. Typically, the liquid preparation is placed in a mould, e.g. such that following sublimation a solid composition, typically in a dosage unit, is formed within the mould. The mould can be an open blister pack whereby the solid dosage unit is formed within the blister pack's depression which is thereafter sealed by a sealing film or foil.

In one embodiment, the process comprises introducing unit dosage quantities of said preparation into depressions of an open blister pack; and then sublimating the preparation to obtain solid dosage forms within said depressions.

The sublimation can be carried out by freeze drying the liquid preparation comprising the first active ingredient, matrix-forming agent(s), controlled release beads and optionally secondary matrix-forming agent(s) in a solvent. In one embodiment, the solvent is water.

The invention thus discloses a process for preparing fast-dispersing dosage forms by lyophilizing a solution, suspension, dispersion or emulsion comprising a combination of a first active ingredient, matrix-forming agent(s), controlled release beads and optionally secondary matrix-forming agent(s). The fast-dispersing dosage form contains a network of the first active ingredient, the matrix-forming agent(s), the controlled release beads and optionally the secondary matrix-forming agent(s), the network having been obtained by sublimating solvent from the liquid preparation that contains these components.

Typically, an initial preparation comprising a first active ingredient, matrix-forming agent(s), controlled release beads and optionally secondary matrix-forming agent(s) in a solvent is prepared, followed by sublimation. The sublimation can be carried out by freeze-drying the preparation. An early dissolution or release of the second active ingredient during preparation of the fast-dispersing dosage form can be prevented by reducing the time of contact between liquid components and the controlled release beads, for example to a period of not more than 45, 35, 25, 20, 15, or 10 minutes, or not more than 5 minutes.

In a freeze-drying procedure, the preparation (in liquid form) that comprises a first active ingredient, matrix-forming agent(s), controlled release beads and any other optional matrix-forming agent(s) in a solvent is filled into moulds. Each mould typically contains a defined amount of such preparation with a defined amount of first active ingredient and a defined amount of beads. In an alternative embodiment, the controlled release beads are pre-filled into moulds in the required amount and optionally cooled and frozen, and subsequently the preparation in liquid form comprising the remaining components of the fast-dispersing dosage form is added into the mould. The preparation in the mould is then frozen, for example by passing gaseous cooling medium over the mould. After the preparation has been frozen, the solvent is sublimated therefrom. The sublimation is carried out in a freeze dryer. In consequence an open matrix network of matrix-forming agent(s) optionally together with other matrix-forming agent(s) included in the preparation, carrying the first active ingredient and the beads, is thereby formed.

The preparation is contained in a mould during the freeze-drying process to produce a solid form in any desired shape. Prior to the lyophilization, the mould may be cooled and frozen (e.g. in a fast-freeze tunnel or on the shelves of the lyophilizer), for example using liquid nitrogen or solid carbon dioxide. In one embodiment, the freezing rate is from 0.1 to 2° C./minute. In another embodiment, the freezing rate is from 0.5 to 1.5° C./minute. In yet another embodiment, the freezing rate is from 10 to 260° C./minute. In another embodiment, the freezing rate is from 20 to 260° C./minute. In a further embodiment, the freezing rate is from 20 to 160° C./minute.

After lyophilization, the freeze dried compositions can either be removed from the mould if desired or stored therein until later use. Typically, each mould is so designed so to produce a unit dosage form of the composition. The composition so obtained is fast-dispersing and disintegrates within at most 30 seconds upon contact with fluid, typically within less than 10 seconds.

The solvent used in the preparation of the composition of the invention is typically water but may optionally also contain a co-solvent (such as an alcohol e.g. tert-butyl alcohol).

The liquid preparation from which the composition of the invention is prepared may contain a pH adjusting agent to adjust the pH thereof within the range of from 2 to 10, typically from 3.5 to 9.5 or from 4.5 to 8. Citric acid and sodium citrate can be used as pH adjusting agent, but others including sodium hydroxide, sodium carbonate, hydrochloric acid and malic acid can also be used. Non-volatile pH adjusting agents will not be removed by freeze drying or other sublimation processes and so may be present in the final composition.

The mould may comprise a series of cylindrical or other shape depressions in it, each of a size corresponding to a desired size of a dosage form to be formed.

In one embodiment, the mould is a depression in a sheet of filmic material. The filmic material may contain more than one depression. The filmic material may be similar to that employed in conventional blister packs which are used for packaging oral tablets and like medicament forms. For example the filmic material may be made of thermoplastic material with the depressions formed by thermoforming or cold forming. Polyvinyl chloride film can be used as filmic material. Laminates of filmic material may also be used.

### EXAMPLES

The invention is further described in the following examples, which are not in any way intended to limit the scope of the invention as claimed.

### Methods

### Method for Testing Matrix Disintegration Time

This test determines the disintegration time of a composition of the invention in an aqueous medium, which is also an indication of its disintegration time in saliva.

Equipment: Electrolab, Model ED2 SAPO

Procedure: The method is followed as per USP 31-NF 26 (General Chapters, <701>Disintegration) and Ph Eur. 1997 (2.9.1. Disintegration of tablets and capsules). Water is filled into the beaker and maintained at 37° C.±0.5° C. using a water bath. The dosage form is placed in a sinker made from copper wire with a diameter of about 0.5 mm (±0.05 mm) and a length of about 15 mm. The sinker is then placed into the basket of the basket rack assembly and the instrument is switched on. The disintegration time is noted in seconds.

### Dissolution Method for Testing Immediate Release of the First Active Ingredient

This test determines the dissolution (%) of the first active ingredient from a composition of the invention in an aqueous medium, which is an indication of the release profile of the first active ingredient.

Equipment: Varian, Model VK7025

Procedure: The method is followed as per USP 32-NF 27 (General Chapters, <711>Dissolution). Dissolution media (0.1N HCl, phosphate buffer pH 6.8, acetate buffer pH 4.5 or 0.5% SLS (sodium lauryl sulfate) in water) are selected on the basis of the active ingredient in the composition. Dissolution bowls are filled with appropriate media volume (500 mL or 900 mL) on the basis of the active ingredient in the composition and the temperature of the medium is maintained at 37° C.±0.5° C. using a water bath. The apparatus used is USP type II (Paddle) and set at 50 rpm. Samples are withdrawn at 5 min, 10 min, 15 min and 30 min. Samples are analyzed chromatographically or by UV, as appropriate, and % release is calculated.

### Dissolution Method for Testing Extended Release of the Second Active Ingredient

This test determines the dissolution (%) of the second active ingredient from a composition of the invention in an aqueous medium, which is an indication of the release profile of the second active ingredient.

Equipment: Varian, Model VK7025

Procedure: The method is followed as per USP 32-NF 27 (General Chapters, <711>Dissolution). Dissolution media (0.1N HCl, phosphate buffer pH 6.8, acetate buffer pH 4.5 or 0.5% SLS in water) are selected on the basis of the active ingredient in the composition. Dissolution bowls are filled with appropriate media volume (900 mL) on the basis of the active ingredient in the composition and the temperature of the medium is maintained at 37° C.±0.5° C. using a water bath. The apparatus used is USP type I (Basket) and set at 100 rpm. Samples are withdrawn at 1 hour, 2 hours, 3 hours, 5 hours, 7 hours, 9 hours and 12 hours. Samples are analyzed chromatographically or by UV, as appropriate, and % release is calculated.

### Method for Measuring Particle Size of the Core

This test determines the particle size of the core using standard sieves (BSS, ASTM). The method follows USP 35-NF 30 (General Chapters, <786> Particle Size Distribution Estimation by Analytical Sieving). The sieves in the range of interest are stacked on top of each other in ascending degrees of coarseness, and the core pellets/beads/particles are placed on the top sieve. The nest of sieves is subjected to a standardized period of agitation, and then the weight of material retained on each sieve is accurately determined.

### Example 1

### Tolterodine ER Particles

| Ingredients | mg/unit |
|---|---|
| Sugar spheres 1 (150-180 μm) | 8.00 |
| Seal coating 2 | |
| Eudragit NE 30 D | 1.60 |
| Talc | 0.80 |
| Purified water | q.s. |
| Drug layer 3 | |
| Tolterodine L-Tartrate | 4.00 |
| HPMC 5 cps | 0.80 |
| Purified water | q.s. |
| ER coating 5 | |
| Eudragit NE 30D | 4.00 |
| HPMC 5 cps | 0.25 |
| Talc | 1.83 |
| Purified water | q.s. |
| Total Weight | 21.28 |

### Brief Manufacturing Procedure:

Tolterodine ER particles were prepared in a fluid bed processor (Wurster coating process). The different components/preparation steps of the ER particles were:
a. Inert core 1: Sugar spheres (Pharm-a-spheres USP/NF, EP) of size range 150-180 μm were selected for seal coating, drug layering and ER coating.
b. Seal coating 2: Eudragit NE 30 D (Colorcon) diluted to 20% w/w concentration was used for the first water insoluble layering. Eudragit NE 30 D dispersion (ration of polymer:talc was 1:0.5) was sprayed on the sugar spheres to obtain a weight gain of 30% w/w.
c. Drug layer 3: An aqueous solution of drug and binder was sprayed onto the sealcoated sugar spheres to a target weight gain of ˜46% w/w. The ratio of tolterodine tartrate:HPMC 5 cps was 5:1.
d. Extended release coating 5: This layer was a combination of Eudragit NE 30 D+HPMC 5 cps+Talc; ˜20% w/w aqueous dispersion of Eudragit NE 30 D+HPMC 5 cps (ratio of Eudragit NE 30 D:HPMC 5 cps was 94.34:5.66; talc: ˜43.1% of polymer content) was sprayed onto the drug-coated particles to obtain a weight gain of 40% w/w.
e. Curing of tolterodine ER particles: The tolterodine ER particles were cured for 12 hours at 40° C.

### Lyophilizate Composition:

| Ingredients | mg/lyophilizate |
|---|---|
| Tolterodine ER particles | 22.72 |
| Levan | 26.00 |
| Mannitol | 21.94 |
| Avicel RC 591 | 8.00 |
| Citric acid (anhydrous) | adjust to pH 4.5 |
| Purified water | q.s. to 400 μl |
| Total weight | 78.66 mg |

### Brief Manufacturing Procedure for Lyophilizates:

1. All excipients were dissolved in purified water to produce the matrix composition. The final volume make up done by purified water and pH adjusted to pH 4.5 using 5% w/v citric acid solution
2. Filling of preformed blister cavities with accurate content of tolterodine ER particles
3. Filling of the matrix solution of step 1 in blister cavities containing tolterodine ER particles
4. Freezing of the filled blisters in liquid nitrogen tunnel
5. Holding of blisters below freezing temperature until whole batch is frozen
6. Loading of frozen blisters into lyophilizer for freeze drying
7. Freeze drying end point monitored by pressure rise test
8. Unloading of dried blisters from the lyophilizer.
9. Sealing of blisters, followed by printing.

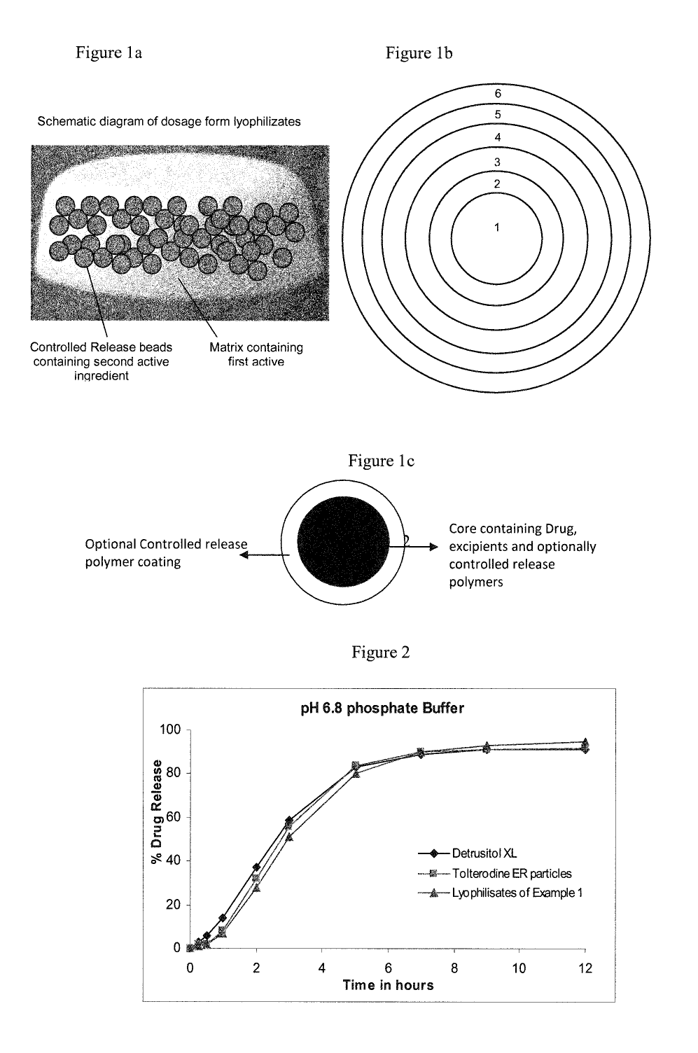

The drug release (dissolution profile) of tolterodine from the tolterodine ER particles and the lyophilizates of Example 1 and, for comparison, from Detrusitol XL in pH 6.8 phosphate buffer is shown in FIG. 2.

### Example 2

### Tolterodine ER Particles

| Ingredients | mg/unit |
|---|---|
| Sugar spheres 1 (150-180μ) | 8.00 |
| Seal coating 2 | |
| Surelease E-7-19010 | 2.40 |
| Purified water | q.s. |
| Drug layer 3 | |
| Tolterodine L-tartrate | 4.00 |
| HPMC 5 cps | 0.80 |
| Purified water | q.s. |
| ER coating 5 | |
| Surelease E-7-19010 | 12.76 |
| HPMC 5 cps | 2.44 |
| Purified water | q.s. |
| Total weight | 30.40 |

### Brief Manufacturing Procedure:

Tolterodine ER particles were prepared in a fluid bed processor (Wurster coating process).

The different components/preparation steps of the ER particles were:
a. Inert core 1: Sugar spheres (Pharm-a-spheres USP/NF, EP) of size range 150-180 μm were selected for seal coating, drug layering and ER coating.
b. Seal coating 2: Surelease (Colorcon) diluted to 15% w/w concentration was used for the first water insoluble layering. Surelease dispersion was sprayed on the sugar spheres to obtain a weight gain of 30% w/w.
c. Drug layer 3: An aqueous solution of drug and binder was sprayed onto the sealcoated sugar spheres to a target weight gain of =46% w/w. The ratio of tolterodine tartrate:HPMC 5 cps was 5:1.
d. Extended telease coating 5: This layer was a combination of Surelease+HPMC 5 cps. ˜15% w/w aqueous dispersion of Surelease+HPMC 5 cps (ratio of Surelease:HPMC 5 cps: 84:16) was sprayed onto the drug coated particles to obtain a weight gain of 100% w/w.
e. Curing of tolterodine ER particles: The tolterodine ER particles were cured for 3 hours at 70° C.

### Lyophilizate Composition:

| Ingredients | mg/lyophilizate |
|---|---|
| Tolterodine ER particles | 31.49 |
| Desmopressin | 0.12 |
| Levan | 26.00 |
| Mannitol | 21.94 |
| Citric acid (anhydrous) | adjust to pH 4.5 |
| Purified water | q.s. to 400 μl |
| Total weight | 79.55 mg |

### Brief Manufacturing Procedure for Lyophilizates:

1. All excipients were dissolved in purified water. The final volume make up done by purified water and pH adjusted to pH 4.5 using 5% w/v citric acid solution.
2. Filling of preformed blister cavities with accurate content of Tolterodine ER particles
3. Filling of desmopressin dispersion in blister cavities containing Tolterodine ER particles.
4. Freezing of the filled blisters in liquid nitrogen tunnel
5. Holding of blisters below freezing temperature until whole batch is frozen
6. Loading of frozen blisters into lyophilizer for freeze drying
7. Drying end point monitored by pressure rise test
8. Unloading of dried blisters from the lyophilizer.
9. Sealing of blisters, followed by printing.

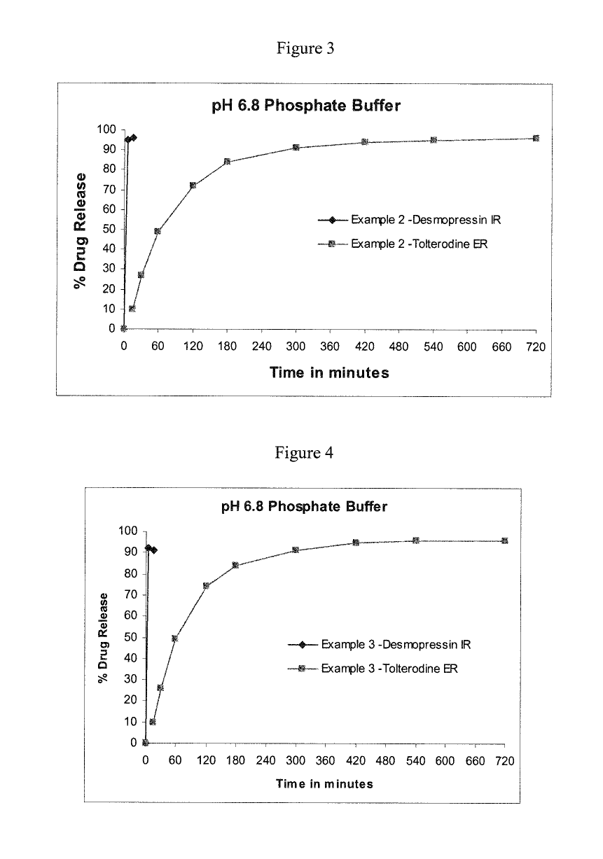

The release of the active ingredients from the dosage form of Example 2 in pH 6.8 phosphate buffer is shown in FIG. 3.

### Example 3

### Tolterodine ER Particles

| Ingredients | mg/unit |
|---|---|
| Sugar spheres 1 (150-180μ) | 8.00 |
| Seal coating 2 | |
| Surelease E-7-19010 | 2.40 |
| Purified water | q.s. |
| Drug layer 3 | |
| Tolterodine L-Tartrate | 4.00 |
| HPMC 5 cps | 0.80 |
| Purified water | q.s. |
| ER coating 5 | |
| Surelease E-7-19010 | 12.76 |
| HPMC 5 cps | 2.44 |
| Purified water | q.s. |
| Total weight | 30.40 |

### Brief Manufacturing Procedure:

Tolterodine ER particles were prepared in a fluid bed processor (Wurster coating process).

The different components/preparation steps of the ER particles were:
a. Inert core 1: Sugar spheres (Pharm-a-spheres USP/NF, EP) of size range 150-180 μm were selected for seal coating, drug layering and ER coating.
b. Seal coating 2: Surelease (Colorcon) diluted to 15% w/w concentration was used for first water insoluble layering. Surelease dispersion was sprayed on the sugar spheres to obtain a weight gain of 30% w/w.
c. Drug layer 3: An aqueous solution of drug and binder was sprayed onto the sealcoated sugar spheres to a target weight gain of =46% w/w. The ratio of Tolterodine Tartrate:HPMC 5 cps was 5:1.
d. Extended release coating 5: This layer was a combination of Surelease+HPMC 5 cps. ˜15% w/w aqueous dispersion of Surelease+HPMC 5 cps (ratio of Surelease:HPMC 5 cps: 84:16) was sprayed onto drug layered particles to obtain a weight gain of 100% w/w.
e. Curing of tolterodine ER particles: Tolterodine ER particles were cured for 3 hours at 70° C.

### Lyophilizate Composition:

| Ingredients | mg/lyophilisate |
|---|---|
| Tolterodine ER particles | 31.49 |
| Desmopressin | 0.12 |
| Levan | 26.00 |
| Mannitol | 21.94 |
| Avicel RC 591 | 4.00 |
| Citric acid (anhydrous) | adjust to pH 4.5 |
| Purified water | q.s. to 400 μl |
| Total weight | 83.55 mg |

### Brief Manufacturing Procedure for Lyophilizate:

1. All excipients dissolved in purified water. The final volume make up done by purified water and pH adjusted to pH 4.5 using 5% w/v citric acid solution.
2. Filling of preformed blister cavities with accurate content of tolterodine ER Particles
3. Filling of desmopressin dispersion in blister cavities containing tolterodine ER Particles.
4. Freezing of the filled blisters in liquid nitrogen tunnel
5. Holding of blisters below freezing temperature until whole batch is frozen
6. Loading of frozen blisters into lyophilizer for freeze drying
7. Drying end point monitored by pressure rise test
8. Unloading of dried blisters from the lyophilizer.
9. Sealing of blisters and followed by printing.

The release of the active ingredients from the dosage form of Example 3 in pH 6.8 phosphate buffer is shown in FIG. 4.

### Example 4

### Tolterodine ER Particles

| Ingredients | mg/unit |
|---|---|
| Sugar Spheres 1 (150-180μ) | 8.00 |
| Seal coating 2 | |
| Surelease E-7-19010 | 2.40 |
| Purified water | q.s. |
| Drug Layering 3 | |
| Tolterodine L-Tartrate | 4.00 |
| HPMC 5 cps | 0.80 |
| Purified water | q.s. |
| ER coating 5 | |
| Surelease E-7-19010 | 12.76 |
| HPMC 5 cps | 2.44 |
| Purified water | q.s. |
| Total weight | 30.40 |

### Brief Manufacturing Procedure:

Tolterodine ER particles were prepared in a fluid bed processor (Wurster coating process).

The different components/preparation steps of ER particles were:
a. Inert core 1: Sugar spheres (Pharm-a-spheres USP/NF, EP) of size range 150-180 μm were selected for seal coating, drug layering and ER coating.
b. Seal coating 2: Surelease (Colorcon) diluted to 15% w/w concentration was used for the first water insoluble layering. Surelease dispersion was sprayed on the sugar spheres to obtain a weight gain of 30% w/w.
c. Drug layer 3: The aqueous solution of drug and binder was sprayed onto the sealcoated sugar spheres to a target weight gain of ˜46% w/w. The ratio of tolterodine tartrate:HPMC 5 cps was 5:1.
d. Extended release coating 5: This layer was a combination of Surelease+HPMC 5 cps. ˜15% w/w aqueous dispersion of Surelease+HPMC 5 cps (ratio of Surelease:HPMC 5 cps: 84:16) was sprayed onto the drug layered particles to obtain a weight gain of 100% w/w
e. Curing of tolterodine ER particles: Tolterodine ER particles were cured for 3 hours at 70° C.

### Lyophilizate Composition:

| Ingredients | mg/lyophilizate |
|---|---|
| Tolterodine ER particles | 31.49 |
| Desmopressin | 0.075 |
| Levan | 26.00 |
| Mannitol | 21.94 |
| Pullulan | 16.00 |
| Citric acid (anhydrous) | adjust to pH 4.5 |
| Purified water | q.s. to 400 μl |
| Total weight | 95.505 mg |

### Brief Manufacturing Procedure for Lyophilizates:

1. All excipients dissolved in purified water. The final volume make up done by purified water and pH adjusted to pH 4.5 using 5% w/v citric acid solutions.
2. Filling of pre formed blister cavities with accurate content of tolterodine ER particles
3. Filling of desmopressin dispersion in blister cavities containing tolterodine ER particles.
4. Freezing of the filled blisters in liquid nitrogen tunnel
5. Holding of blisters below freezing temperature until whole batch is frozen
6. Loading of frozen blisters into lyophilizer for freeze drying
7. Drying end point monitored by pressure rise test
8. Unloading of dried blisters from the lyophilizer.
9. Sealing of blisters, followed by printing.

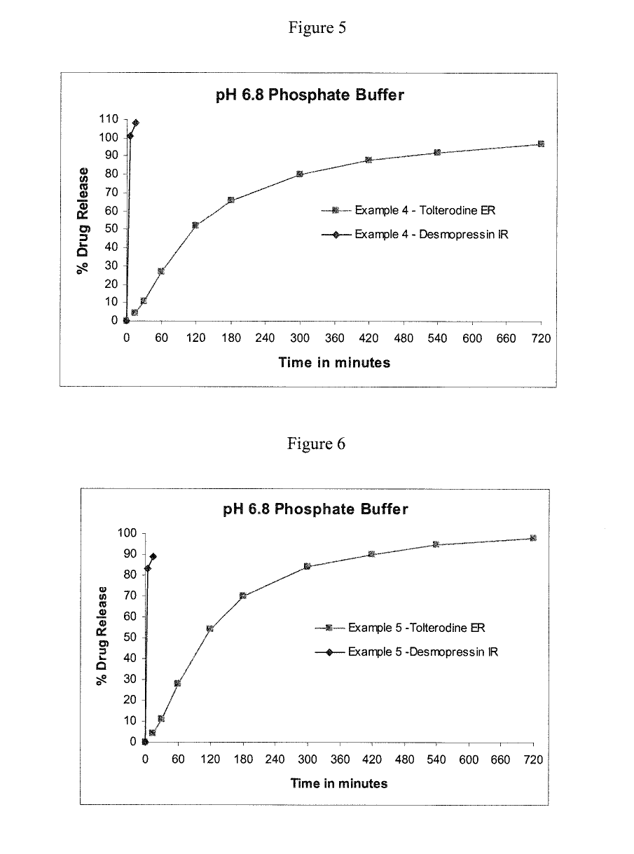

The release of the active ingredients from the dosage form of Example 4 in pH 6.8 phosphate buffer is shown in FIG. 5.

### Example 5

### Tolterodine ER Particles

| Ingredients | mg/unit |
|---|---|
| Sugar spheres 1 (150-180μ) | 8.00 |
| Seal coating 2 | |
| Surelease E-7-19010 | 2.40 |
| Purified water | q.s. |
| Drug layer 3 | |
| Tolterodine L-tartrate | 4.00 |
| HPMC 5 cps | 0.80 |
| Purified water | q.s. |
| ER coating 5 | |
| Surelease E-7-19010 | 12.76 |
| HPMC 5 cps | 2.44 |
| Purified water | q.s. |
| Total weight | 30.40 |

### Brief Manufacturing Procedure:

Tolterodine ER particles were prepared in a fluid bed processor (Wurster coating process).

The different components/preparation steps of the ER particles were:
a. Inert core 1: Sugar spheres (Pharm-a-spheres USP/NF, EP) of size range 150-180 μm were selected for seal coating, drug layering and ER coating.
b. Seal coating 2: Surelease (Colorcon) diluted to 15% w/w concentration was used for first water insoluble layering. Surelease dispersion was sprayed on sugar spheres to obtain a weight gain of 30% w/w.
c. Drug layer 3: The aqueous solution of drug-binder was sprayed onto the sealcoated sugar spheres to a target weight gain of ˜46% w/w. The ratio of Tolterodine Tartrate:HPMC 5 cps was 5:1.
d. Extended Release coating 5: This layer was a combination of Surelease+HPMC 5 cps. ˜15% w/w aqueous dispersion of Surelease+HPMC 5 cps (ratio of Surelease:HPMC 5 cps: 84:16) was sprayed onto drug layered particles to obtain a weight gain of 100% w/w
e. Curing of Tolterodine ER particles: Tolterodine ER particles were cured for 3 hours at 70° C.

### Lyophilizate Composition:

| Ingredients | mg/lyophilizate |
|---|---|
| Tolterodine ER particles | 31.49 |
| Desmopressin | 0.075 |
| Levan | 26.00 |
| Mannitol | 21.94 |
| Acacia | 10.00 |
| Citric acid (anhydrous) | adjust to pH 4.5 |
| Purified water | q.s. to 400 μl |
| Total weight | 89.505 mg |

### Brief Manufacturing Procedure for Lyophilizates:

1. All excipients dissolved in purified water. The final volume make up done by purified water and pH adjusted to pH 4.5 using 5% w/v citric acid solutions.
2. Filling of pre formed blister cavities with accurate content of tolterodine ER particles
3. Filling of desmopressin dispersion in blister cavities containing Tolterodine ER Particles.
4. Freezing of the filled blisters in liquid nitrogen tunnel
5. Holding of blisters below freezing temperature until whole batch is frozen
6. Loading of frozen blisters into lyophilizer for freeze drying
7. Drying end point monitored by pressure rise test
8. Unloading of dried blisters from the lyophilizer.
9. Sealing of blisters and followed by printing.

The release of the active ingredients from the dosage form of Example 5 in pH 6.8 phosphate buffer is shown in FIG. 6.

### Example 6

### Tolterodine ER Particles

| Ingredients | mg/unit |
|---|---|
| Sugar spheres 1 (106-125μ) | 8.00 |
| Seal coating 2 | |
| Surelease E-7-19010 | 2.40 |
| Purified water | q.s. |
| Seal coated pellets | 10.40 |
| Drug layer 3 | |
| Tolterodine L-tartrate | 4.00 |
| HPMC 5 cps | 0.80 |
| Purified water | q.s. |
| Total weight | 15.20 |
| ER coating 5 | |
| Surelease E-7-19010 | 15.32 |
| HPMC 5 cps | 2.92 |
| Purified water | q.s. |
| Total weight | 33.44 |
| Coat 6 | |
| Surelease E-7-19010 | 7.47 |
| Eudragit L100 | 0.912 |
| HPMC 5 cps | 0.729 |
| Purified water | q.s. |
| Total weight | 42.55 |

### Brief Manufacturing Procedure:

Tolterodine ER particles were prepared in fluid bed processor (Wurster coating process).

The different components/preparation steps of ER particles were:
a. Inert core 1: Sugar spheres (Pharm-a-spheres USP/NF, EP) of size range 106-125 μm were selected for seal coating, drug layering and ER coating.
b. Seal coating 2: Surelease (Colorcon) diluted to 15% w/w concentration was used for the first water insoluble layering. Surelease dispersion was sprayed on the sugar spheres to obtain a weight gain of 30% w/w.
c. Drug layer 3: The aqueous solution of drug and binder was sprayed onto the sealcoated sugar spheres to a target weight gain of ˜46% w/w. The ratio of tolterodine tartrate:HPMC 5 cps was 5:1.
d. Extended release coating 5: This layer was a combination of Surelease+HPMC 5 cps. ˜15% w/w aqueous dispersion of Surelease+HPMC 5 cps (ratio of Surelease:HPMC 5 cps: 84:16) was sprayed onto drug layered particles to obtain a weight gain of 120% w/w.
e. Curing of tolterodine ER particles: Tolterodine ER particles were cured for 3 hours at 70° C.
f. Coating 6: This layer was a combination of Surelease+HPMC 5 cps+Eudragit L100. ˜15 w/w aqueous dispersion of Surelease:Eudragit L100:HPMC 5 cps 82:10:8 was sprayed onto the tolterodine ER particles to obtain a weight gain of 60% w/w.
g. Curing of tolterodine particles: Coated particles were cured for 3 hours at 70° C.

### Lyophilizate Composition:

| Ingredients | mg/unit |
|---|---|
| Tolterodine particles | 43.95 |
| Levan | 26.00 |
| Mannitol | 21.94 |
| Avicel RC 591 | 8.00 |
| Citric acid (anhydrous) | adjust to pH 4.5 |
| Purified water | q.s. to 400 μl |
| Total weight | 99.89 |

### Brief Manufacturing Procedure for Lyophilizate:

1. All excipients were dissolved in purified water to produce the matrix composition. The final volume make up done by purified water and pH adjusted to pH 4.5 using 5% w/v citric acid solutions.
2. Filling of pre-formed blister cavities with accurate content of tolterodine particles.
3. Filling the matrix solution of step 1 in blister cavities containing tolterodine particles.
4. Freezing of the filled blisters in liquid nitrogen tunnel
5. Holding of blisters below freezing temperature until whole batch is frozen
6. Loading of frozen blisters into lyophilizer for freeze drying
7. Drying end point monitored by pressure rise test
8. Unloading of dried blisters from the lyophilizer.
9. Sealing of blisters and followed by packing.

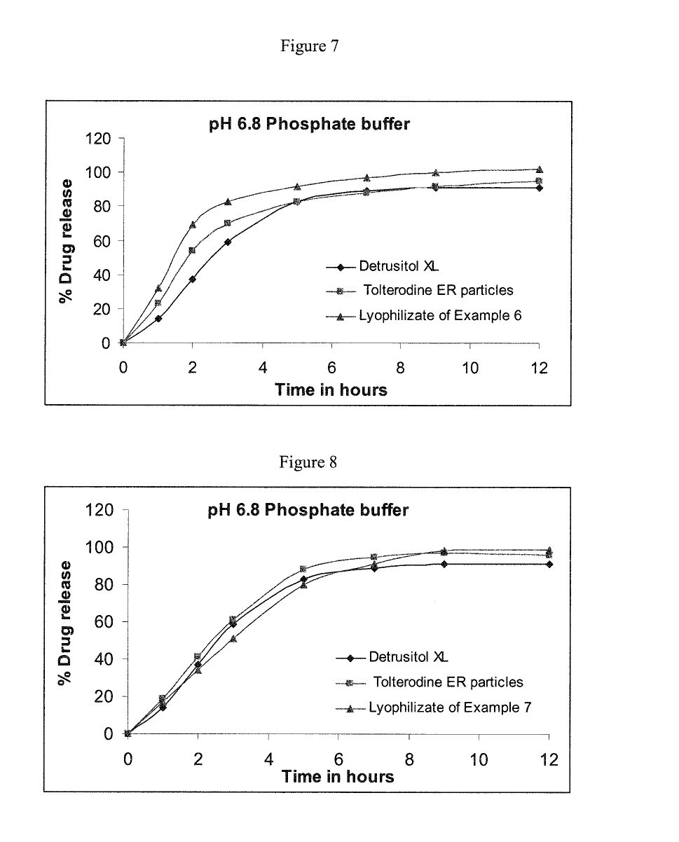

The drug release (dissolution profile) of tolterodine from the tolterodine ER particles and the lyophilizates of Example 6 and, for comparison, from Detrusitol XL in pH 6.8 phosphate buffer is shown in FIG. 7.

### Example 7

### Tolterodine ER Particles

| Ingredients | mg/unit |
|---|---|
| Microcrystalline cellulose | 21.1 |
| spheres 1 (150-200 μm) | |
| Drug layer 3 | |
| Tolterodine L-tartrate | 4.0 |
| HPMC 5 cps | 0.8 |
| Talc | 0.8 |
| Purified water | q.s. |
| ER coating 5 | |
| Eudragit NE 30D | 3.6 |
| HPMC 5 cps | 0.2 |
| Talc | 1.5 |
| Purified water | q.s. |
| Total weight | 32.0 |

### Brief Manufacturing Procedure:

Tolterodine ER particles were prepared in a fluid bed processor (Wurster coating process).

The different components/preparation steps of ER particles were:
a. Inert core 1: Microcrystalline cellulose spheres (Cellets 175) of size range 150-200 μm were selected for drug layering and ER coating.
b. Drug layer 3: The aqueous dispersion of drug and talc in binder solution was sprayed onto the cores to a target weight gain of =27% w/w. The ratio of tolterodine tartrate:HPMC 5 cps was 5:1.
c. Extended release coating 5: This layer was a combination of Eudragit NE 30 D, HPMC 5 cps and talc. The aqueous dispersion of Eudragit NE 30 D, HPMC 5 cps and talc was sprayed on the drug layered particles to a target weight gain of ˜20% w/w.
d. Curing of tolterodine ER particles: Tolterodine ER particles were cured for 24 hours at 40° C.

### Lyophilizate Composition:

| Ingredients | Per Unit (mg) |
|---|---|
| Tolterodine ER pellets | 32.77 |
| Levan | 26.00 |
| Mannitol (Perlitol 160C) | 21.94 |
| Avicel RC 591 | 8.00 |
| Citric acid Sol. (5% w/v) | q.s to pH 4.5 |
| Purified water | q.s to 400 μl |
| Total weight | 88.71 mg |

### Brief Manufacturing Procedure for Lyophilizate:

1. All excipients were dissolved in purified water. The final volume make up done by purified water and pH adjusted to pH 4.5 using 5% w/v citric acid solutions.
2. Filling of preformed blister cavities with accurate content of tolterodine ER particles
3. Filling of the matrix solution of step 1 in blister cavities containing tolterodine ER particles.
4. Freezing of the filled blisters in liquid nitrogen tunnel
5. Holding of blisters below freezing temperature till whole batch is frozen
6. Loading of frozen blisters into lyophilizer for freeze drying
7. Freeze drying end point monitored by pressure rise test
8. Unloading of dried blisters from the lyophilizer.
9. Sealing of blisters and followed by printing.

The drug release (dissolution profile) of tolterodine from the tolterodine ER particles and the lyophilizates of Example 7 and, for comparison, from Detrusitol XL in pH 6.8 phosphate buffer is shown in FIG. 8.

### Example 8

### Tolterodine ER Particles

| Ingredients | mg/unit |
|---|---|
| Microcrystalline cellulose | 21.1 |
| spheres 1 (150-200 μm) | |
| Drug layer 3 | |
| Tolterodine L-tartrate | 4.0 |
| HPMC 5 cps | 0.8 |
| Talc | 0.8 |
| Purified water | q.s. |
| ER coating 5 | |
| Eudragit NE 30D | 3.6 |
| HPMC 5 cps | 0.2 |
| Talc | 1.5 |
| Purified water | q.s. |
| coating 6 | |
| Eudragit L 30D-55 | 4.0 |
| Talc | 2.0 |
| Triethylcitrate | 0.4 |
| Total weight | 38.4 |

### Brief Manufacturing Procedure:

Tolterodine particles were prepared in a fluid bed processor (Wurster coating process).

The different components/preparation steps of the particles were:
a. Inert core 1: Microcrystalline cellulose spheres (Cellets 175) of size range 150-200 μm were selected for drug layering and ER coating.
b. Drug layer 3: The aqueous dispersion of drug and talc in binder solution was sprayed onto the cores to a target weight gain of ˜27% w/w. The ratio of tolterodine tartrate:HPMC 5 cps was 5:1.
c. Extended release coating 5: This layer was a combination of Eudragit NE 30 D, HPMC 5 cps and talc. The aqueous dispersion of Eudragit NE 30 D, HPMC 5 cps and talc was sprayed on the drug layered particles to a target weight gain of 20% w/w.
d. Curing of tolterodine ER particles: Tolterodine ER particles were cured for 24 hours at 40° C.
e. Coating 6: This layer was a combination of Eudragit L 30D-55, talc and triethylcitrate. The aqueous dispersion of Eudragit L 30D-55, talc and triethylcitrate was sprayed on the extended release coated particles to a target weight gain of ˜20% w/w.
f. Curing of particles: Tolterodine coated particles were cured for 2 hours at 40° C.

### Lyophilizate Composition:

| Ingredients | Per Unit (mg) |
|---|---|
| Tolterodine ER pellets | 38.94 |
| Levan | 26.00 |
| Mannitol (Perlitol 160C) | 21.94 |
| Avicel RC 591 | 8.00 |
| Citric acid Sol. (5% w/v) | q.s to pH 4.5 |
| Purified water | q.s to 400 μl |
| Total weight | 94.88 mg |

### Brief Manufacturing Procedure for Lyophilizate:

1. All excipients were dissolved in purified water. The final volume make up done by purified water and pH adjusted to pH 4.5 using 5% w/v citric acid solutions.
2. Filling of preformed blister cavities with accurate content of tolterodine ER particles
3. Filling of the matrix solution of step 1 in blister cavities containing tolterodine ER particles.
4. Freezing of the filled blisters in liquid nitrogen tunnel
5. Holding of blisters below freezing temperature till whole batch is frozen
6. Loading of frozen blisters into lyophilizer for freeze drying
7. Freeze drying end point monitored by pressure rise test
8. Unloading of dried blisters from the lyophilizer.
9. Sealing of blisters and followed by printing.

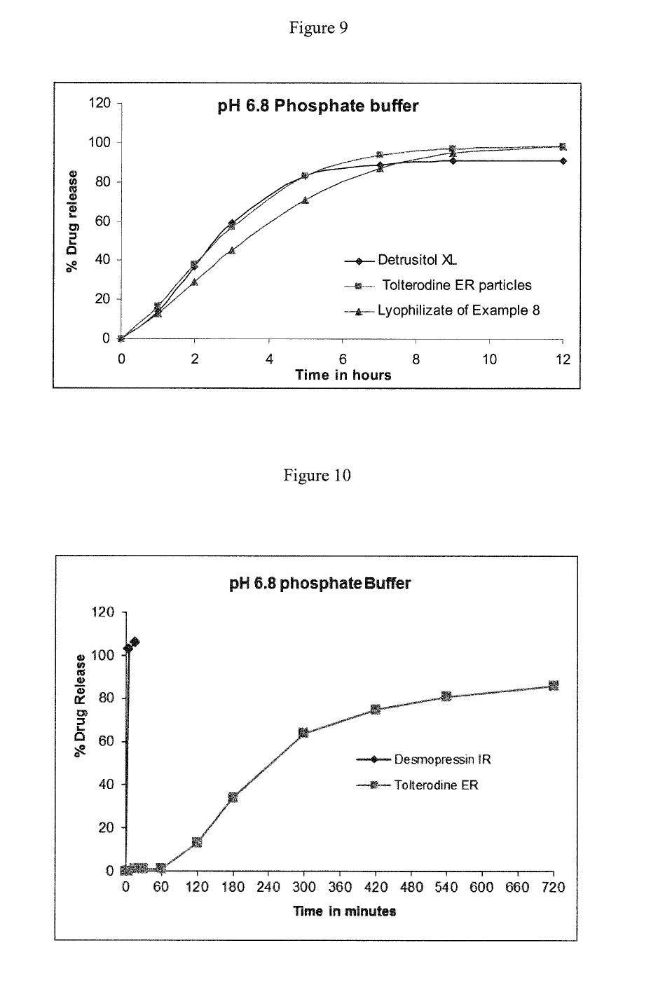

The drug release (dissolution profile) of tolterodine from the tolterodine ER particles and the lyophilizates of Example 8 and, for comparison, from Detrusitol XL in pH 6.8 phosphate buffer is shown in FIG. 9.

### Example 9

### Tolterodine ER Particles

| Ingredients | mg/unit |
|---|---|
| Sugar Spheres 1 (150-180μ) | 8.00 |
| Seal coating 2 | |
| Surelease E-7-19010 | 2.40 |
| Purified water | q.s. |
| Drug Layering 3 | |
| Tolterodine L-Tartrate | 4.00 |
| HPMC 5 cps | 0.80 |
| Purified water | q.s. |
| Outer (barrier) seal coating 4 | |
| HPMC 5 cps | 1.52 |
| Purified water | q.s. |
| ER coating 5 | |
| Eudragit NE30D | 4.49 |
| HPMC 5 cps | 0.26 |
| Talc | 1.93 |
| Purified water | q.s. |
| Outer coat 6 | |
| Eudragit NE30D | 5.85 |
| Talc | 5.85 |
| Purified water | q.s. |
| Total weight | 35.1 |

### Brief Manufacturing Procedure:

Tolterodine ER particles were prepared in fluid bed processor (Wruster coating process).

The different components/preparation steps of the ER particles were:
a. Inert core 1: Sugar spheres (Pharm-a-spheres USP/NF, EP) of size range 150-180 μm were selected for seal coating, drug layering, barrier coating, ER coating and outer coating.
b. Seal coating 2: Surelease (Colorcon) diluted to 15% w/w concentration was used for the first water insoluble layering. Surelease dispersion was sprayed onto sugar spheres to obtain the weight gain 30% w/w.
c. Drug layer 3: The aqueous solution of drug-binder was sprayed onto the seal coated sugar spheres to target weight gain ˜46% w/w. The ratio of Tolterodine Tartrate:HPMC 5 cps was 5:1.
d. Outer (barrier) seal coating 4: The aqueous solution of HPMC 5 cps (5% w/v) was sprayed onto the drug layered and seal coated sugar spheres to target weight gain ˜10% w/w.
e. Extended Release coating 5: This layer was combination of Eudragit NE30D+HPMC 5 cps+Talc. ˜20% w/w aqueous dispersion of Eudragit NE30D+HPMC 5 cps+Talc (Ratio of Eudragit NE30D:HPMC 5 cps; 94.34:5.66 and Talc is 43.1% of polymer content) was sprayed on to the barrier layered particles to obtain the weight gain 40% w/w.
f. Outer coating 6: This layer was a combination of Eudragit NE30D+Talc. ˜20% w/w aqueous dispersion of Eudragit NE30D+Talc (Ratio of Eudragit NE30D:Talc; 50:50) was sprayed onto the ER layered particles to obtain the weight gain w/w.
g. Curing of Tolterodine ER particles: Tolterodine ER particles were cured for 12 hours at 40° C.

### Lyophilisates Composition:

| Ingredients | mg/unit |
|---|---|
| Tolterodine ER Particles | 36.69* |
| Desmopressin | 0.12 |
| Levan | 26.00 |
| Mannitol | 21.94 |
| Avicel RC 591 | 8.00 |
| Citric acid (anhydrous) | adjust to pH 4.50 |
| P. water | q.s. to 400 μl |
| Total weight | 92.75 mg |

### Brief Manufacturing Procedure for Lyophilisates:

1. All excipients were dissolved or dispersed in purified water. The final volume make up was done by purified water and pH adjusted to pH 4.5 using 5% w/v citric acid solutions.
2. Filling of pre-formed blister cavities with accurate content of tolterodine ER particles.
3. Filling of desmopressin dispersion in blister cavities containing tolterodine ER particles.
4. Freezing of the filled blisters in liquid nitrogen tunnel
5. Holding of blisters below freezing temperature till whole batch is frozen.
6. Loading of frozen blisters into lyophilizer for freeze drying.
7. Drying end point monitored by pressure rise test.
8. Unloading of dried blisters from the lyophilizer.
9. Sealing of blisters and followed by printing.

The release of the active ingredients from the lyophilisate dosage form of example 9 in pH 6.8 phosphate buffer is shown in FIG. 10.

### Example 10

### Lyophilisates Composition

| Ingredients | mg/unit |
|---|---|
| Tolterodine ER Particles of Example 9 | 36.69* |
| Desmopressin | 0.12 |
| Inulin | 48.00 |
| Mannitol | 20.00 |
| Sodium alginate (Keltone LVCR) | 4.00 |
| Citrate buffer | q.s pH 4.30 |
| Purified water | q.s. to 400 μl |
| Total weight | 108.81 mg |

### Brief Manufacturing Procedure for Lyophilisates:

1. All excipients were dissolved in purified water. The final volume make up done by purified water and pH adjusted to pH 4.5 using 5% w/v citric acid solutions.
2. Filling of pre-formed blister cavities with accurate content of tolterodine ER particles.
3. Filling of desmopressin dispersion in blister cavities containing tolterodine ER particles.
4. Freezing of the filled blisters in liquid nitrogen tunnel.
5. Holding of blisters below freezing temperature till whole batch is frozen.
6. Loading of frozen blisters into lyophilizer for freeze drying.
7. Drying end point monitored by pressure rise test.
8. Unloading of dried blisters from the lyophilizer.
9. Sealing of blisters and followed by printing.

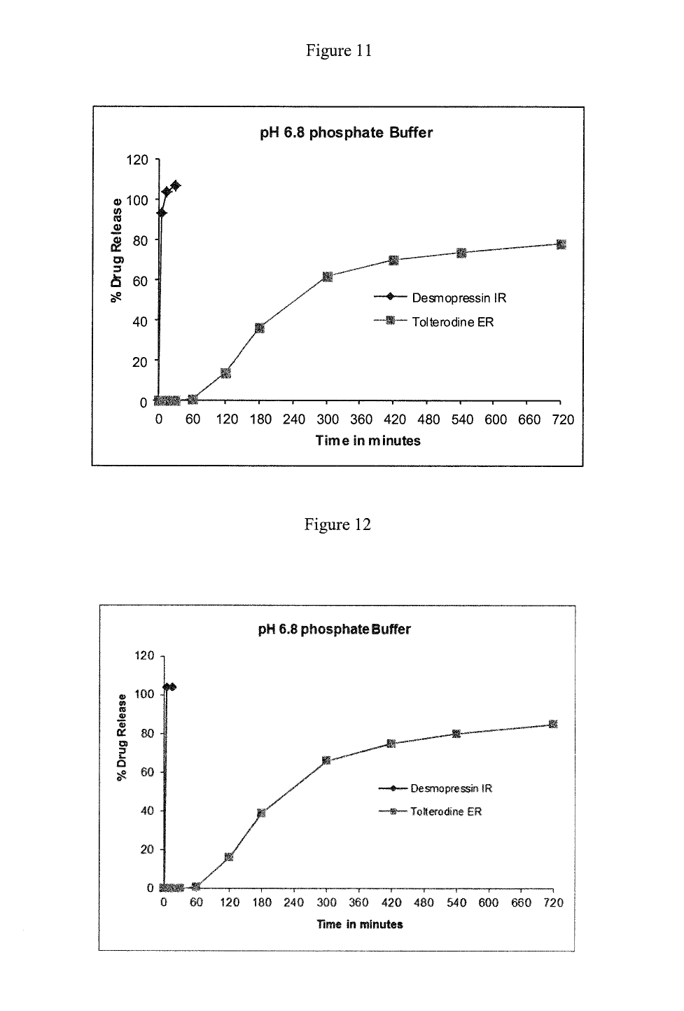

The release of the active ingredients from the lyophilizate dosage form of example 10 in pH 6.8 phosphate buffer is shown in FIG. 11.

### Example 11

### Lyophilisates Composition

| Ingredients | mg/unit |
|---|---|
| Tolterodine ER Particles (Example 9) | 36.69* |
| Desmopressin | 0.12 |
| Inulin | 48.00 |
| Mannitol | 20.00 |
| HPMC 3 cps | 2.00 |
| Citrate buffer | q.s pH 4.30 |
| P. water | q.s. to 400 μl |
| Total weight | 106.81 mg |

### Brief Manufacturing Procedure for Lyophilisates:

1. All excipients were dissolved in purified water. The final volume make up was done by purified water and pH adjusted to pH 4.5 using 5% w/v citric acid solutions.
2. Filling of pre-formed blister cavities with accurate content of tolterodine ER particles.
3. Filling of desmopressin dispersion in blister cavities containing tolterodine ER particles.
4. Freezing of the filled blisters in liquid nitrogen tunnel.
5. Holding of blisters below freezing temperature till whole batch is frozen.
6. Loading of frozen blisters into lyophilizer for freeze drying.
7. Drying end point monitored by pressure rise test.
8. Unloading of dried blisters from the lyophilizer.
9. Sealing of blisters and followed by printing.

The release of the active ingredients from the lyophilizate dosage form of example 11 in pH 6.8 phosphate buffer is shown in FIG. 12.

### Example 12

### Lyophilisates Composition

| Ingredients | mg/unit |
|---|---|
| Tolterodine ER Particles of Example 9 | 36.69* |
| Desmopressin | 0.12 |
| Inulin | 48.00 |
| Mannitol | 20.00 |
| Citrate buffer | q.s pH 4.30 |
| P. water | q.s. to 400 μl |
| Total weight | 104.81 mg |

### Brief Manufacturing Procedure for Lyophilisates:

1. All excipients were dissolved in purified water. The final volume make up was done by purified water and pH adjusted to pH 4.5 using 5% w/v citric acid solutions.
2. Filling of pre-formed blister cavities with accurate content of tolterodine ER particles.
3. Filling of desmopressin dispersion in blister cavities containing tolterodine ER particles.
4. Freezing of the filled blisters in liquid nitrogen tunnel.
5. Holding of blisters below freezing temperature till whole batch is frozen.
6. Loading of frozen blisters into lyophilizer for freeze drying.
7. Drying end point monitored by pressure rise test.
8. Unloading of dried blisters from the lyophilizer.
9. Sealing of blisters and followed by printing.

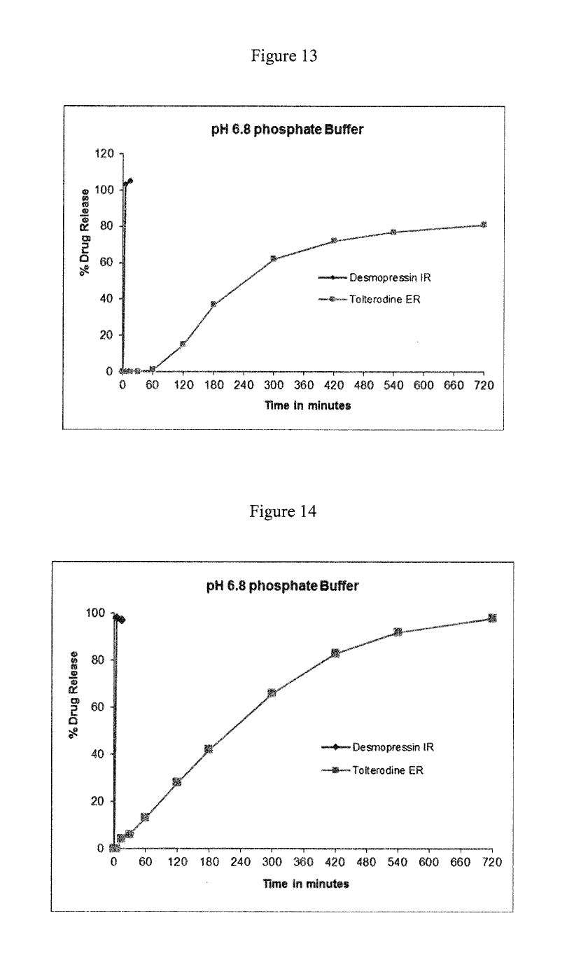

The release of the active ingredients from the lyophilizate dosage form of example 12 in pH 6.8 phosphate buffer is shown in FIG. 13.

### Example 13

### Tolterodine ER particles

| Ingredients | mg/unit |
|---|---|
| MCC pellets (Cellets 175) - 1 (150-200μ) | 21.00 |
| Drug Layering 3 | |
| Tolterodine L-Tartrate | 4.00 |
| HPMC 5 cps | 0.80 |
| Talc | 0.80 |
| Purified water | q.s. |
| ER coating 5 | |
| Eudragit NE30D | 3.57 |
| HPMC 5 cps | 0.21 |
| Talc | 1.54 |
| Purified water | q.s. |
| Outer coat 6 | |
| Eudragit L30 D55 | 4.00 |
| TEC | 0.40 |
| Talc | 2.00 |
| Purified water | q.s. |
| Total weight | 38.32 |

### Brief Manufacturing Procedure:

Tolterodine ER particles were prepared in fluid bed processor (Wurster coating process).

The different components/preparation steps of ER particles were:
1. Inert core 1: MCC Pellets (Cellets 175) of size range 150-200 μm were selected as starting material for drug layering followed by ER coating.
2. Drug layer 3: The aqueous dispersion of drug-binder was sprayed onto the MCC pellets to target the weight gain ˜26.58% w/w. The ratio of Tolterodine Tartrate:HPMC 5 cps was 5:1.
3. Extended Release coating 5: This layer was a combination of Eudragit NE30D+HPMC 5 cps+Talc. ˜20% w/w aqueous dispersion of Eudragit NE30D+HPMC 5 cps+Talc (Ratio of Eudragit NE30D:HPMC 5 cps; 94.34:5.66 and Talc is 43.1% of polymer content) was sprayed on to drug layered particles to obtain the weight gain 20% w/w.
4. Outer coating 6: This layer was combination of Eudragit L30 D55+TEC+Talc. ˜20% w/w aqueous dispersion of Eudragit L30 D55+TEC+Talc (Eudragit L30 D55: Talc was 1:0.5 whereas TEC was 10% of actual Eudragit polymer content) was sprayed onto the ER particles to obtain the weight gain 20% w/w.
5. Curing of Tolterodine ER particles: Tolterodine ER particles were cured for 24 hours at 40° C. after blending with 2% Talc as external phase.

### Lyophilisates Composition:

| Ingredients | mg/unit |
|---|---|
| Tolterodine ER Particles | 38.94 |
| Desmopressin | 0.12 |
| Levan | 26.00 |
| Mannitol | 21.94 |
| Citric acid (anhydrous) | adjust to pH 4.5 |
| P. water | q.s. to 400 μl |
| Total weight | 87.00 |

### Brief Manufacturing Procedure for Lyophilisates:

1. All excipients were dissolved in purified water. The final volume make up was done by purified water and pH adjusted to pH 4.5 using 5% w/v citric acid solutions.
2. Filling of pre-formed blister cavities with accurate content of tolterodine ER particles.
3. Filling of desmopressin dispersion in blister cavities containing tolterodine ER particles.
4. Freezing of the filled blisters in liquid nitrogen tunnel.
5. Holding of blisters below freezing temperature till whole batch is frozen.
6. Loading of frozen blisters into lyophilizer for freeze drying.
7. Drying end point monitored by pressure rise test.
8. Unloading of dried blisters from the lyophilizer.
9. Sealing of blisters and followed by printing.

The release of the active ingredients from the lyophilizate dosage form of example 13 in pH 6.8 phosphate buffer is shown in FIG. 14.

### Example 14

### Tolterodine ER particles

| Ingredients | mg/unit |
|---|---|
| MCC pellets (Cellets 175)(150-200μ) - 1 | 21.00 |
| Drug Layering 3 | |
| Tolterodine L-Tartrate | 4.00 |
| HPMC 5 cps | 0.80 |
| Talc | 0.80 |
| Purified water | q.s. |
| ER coating 5 | |
| Eudragit NE30D | 3.57 |
| HPMC 5 cps | 0.21 |
| Talc | 1.54 |
| Purified water | q.s. |
| Total Weight | 31.92 |

### Brief Manufacturing Procedure:

Tolterodine ER particles were prepared in fluid bed processor (Wurster coating process).

The different components/preparation steps of ER particles were:
1. Inert core 1: MCC Pellets (Cellets 175) of size range 150-200 μm were selected as starting material for drug layering followed by ER coating.
2. Drug layer 3: The aqueous dispersion of drug-binder was sprayed onto the MCC pellets to target the weight gain ˜26.58% w/w. The ratio of Tolterodine Tartrate:HPMC 5 cps was 5:1.
3. Extended Release coating 5: This layer was combination of Eudragit NE30D+HPMC 5 cps+Talc. ˜20 w/w aqueous dispersion of Eudragit NE30D+HPMC 5 cps+Talc (Ratio of Eudragit NE30D:HPMC 5 cps; 94.34:5.66 and Talc is 43.1% of polymer content) was sprayed on to drug layered particles to obtain the weight gain 20% w/w.
4. Curing of Tolterodine ER particles: Tolterodine ER particles were cured for 24 hours at 40° C. after blending with 2% Talc as external phase.

### Lyophilisates Composition:

| Ingredients | mg/unit |
|---|---|
| Tolterodine ER Particles | 32.77 |
| Desmopressin | 0.12 |
| Levan | 26.00 |
| Mannitol | 21.94 |
| Maltodextrin | 30.00 |
| Citric acid (anhydrous) | adjust to pH 4.5 |
| P. water | q.s. to 400 μl |
| Total weight | 110.83 |

### Brief Manufacturing Procedure for Lyophilisates:

1. All excipients were dissolved in purified water. The final volume make up was done by purified water and pH adjusted to pH 4.5 using 5% w/v citric acid solutions.
2. Filling of pre-formed blister cavities with accurate content of tolterodine ER particles.
3. Filling of desmopressin dispersion in blister cavities containing tolterodine ER particles.
4. Freezing of the filled blisters in liquid nitrogen tunnel.
5. Holding of blisters below freezing temperature till whole batch is frozen.
6. Loading of frozen blisters into lyophilizer for freeze drying.
7. Drying end point monitored by pressure rise test.
8. Unloading of dried blisters from the lyophilizer.
9. Sealing of blisters and followed by printing.

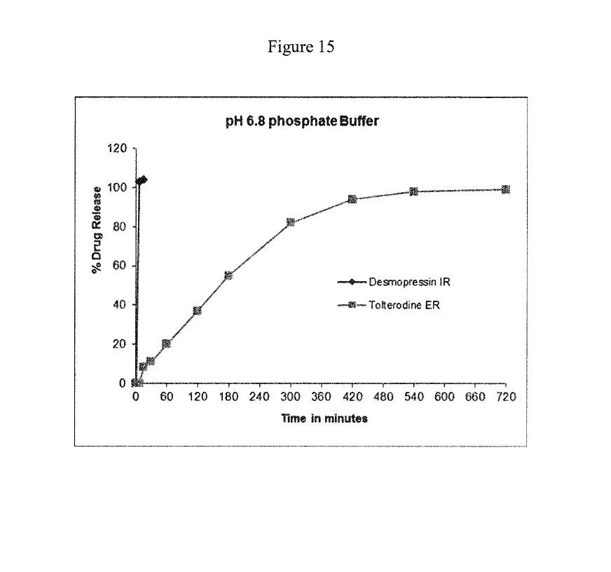

The release of the active ingredients from the lyophilizate dosage form of example 14 in pH 6.8 phosphate buffer is shown in FIG. 15.

1. A pharmaceutical composition comprising
an open matrix network comprising a first pharmaceutically active ingredient;
one or more matrix-forming agents; and
controlled release beads comprising a second pharmaceutically active ingredient.

2. The pharmaceutical composition according to claim 1, wherein the one or more matrix-forming agents are selected from the group consisting of levan, inulin, pullulan, HPMC, maltodextrin, acacia, sodium alginate, and combinations thereof.

3. The pharmaceutical composition according to claim 1, wherein the open matrix network further comprises mannitol, trehalose, and/or raffinose.

4. The pharmaceutical composition according to claim 1, which wherein the composition dissolves in a standardized aqueous medium within 30 seconds.

5. The pharmaceutical composition according to claim 4, wherein the composition dissolves in a standardized aqueous medium within 10 seconds.

6. The pharmaceutical composition according to claim 1, wherein the first pharmaceutically active ingredient is desmopressin acetate.

7. The pharmaceutical composition according to claim 1, wherein the controlled release beads comprise a core (1) of a water-soluble, water-insoluble, or water-swellable inert material having
(i) on the core (1) an optional inner sealcoat layer (2) of a substantially water-insoluble or substantially water-soluble polymer;
(ii) an inner drug-containing layer (3) covering the core (1) or inner sealcoat layer (2) and containing the second active ingredient; and
(iii) on the inner drug-containing layer (3) an outer membrane layer (5) of polymer effective for controlled release of the second active ingredient from the inner drug-containing layer (3).

8. The pharmaceutical composition according to claim 7, wherein the core (1) is a water-soluble sugar sphere.

9. The pharmaceutical composition according to claim 7, wherein the core (1) is a water-swellable microcrystalline cellulose core.

10. The pharmaceutical composition according to claim 7, wherein the amount of the inner sealcoat layer (2) ranges from about 4 to about 15% (w/w) of the controlled release bead.

11. The pharmaceutical composition according to claim 7, wherein the amount of the inner drug-containing layer (3) ranges from about 5 to about 25% (w/w) of the controlled release bead.

12. The pharmaceutical composition according to claim 7, wherein the amount of the outer membrane layer (5) ranges from about 25 to about 55% (w/w) of the controlled release bead.

13. The pharmaceutical composition according to claim 7, wherein the outer membrane layer (5) is coated with an additional polymer layer (6) of a coating with pH-dependant permeability.

14. The pharmaceutical composition according to claim 7, wherein the substantially water-insoluble polymer of the inner sealcoat layer (2) comprises ethyl cellulose.

15. The pharmaceutical composition according to claim 7, wherein the inner drug-containing layer (3) comprises hydroxypropylmethyl cellulose as binder.

16. The pharmaceutical composition according to claim 7, wherein the outer membrane layer (5) effective for controlled release of the second active ingredient comprises a combination of hydroxypropylmethyl cellulose and ethyl cellulose.

17. The pharmaceutical composition according to claim 1, wherein the second active ingredient is an antimuscarinic compound.

18. The pharmaceutical composition according to claim 17, wherein the anti-muscarinic compound is selected from the group consisting of tolterodine, 5-hydroxymethyl metabolite of tolterodine, (S)-enantiomer of tolterodine, 5-hydroxymethyl metabolite of the (S)-enantiomer of tolterodine, racemate of tolterodine, its prodrug forms thereof, and pharmacologically acceptable salts thereof.

19. The pharmaceutical composition according to claim 18, wherein the anti-muscarinic compound is tolterodine or a pharmacologically acceptable salt thereof.

20. The pharmaceutical composition according to claim 19, wherein the anti-muscarinic compound is tolterodine tartrate.

21. The pharmaceutical composition according to claim 20, wherein tolterodine tartrate releases in vitro from about 0% to about 40% after 1 hour, from about 35% to about 85% after 3 hours, and from about 65% to about 100% after 7 hours.

22. The pharmaceutical composition according to claim 17, wherein the composition is for the treatment of overactive bladder.

23. The pharmaceutical composition according to claim 17, of wherein the composition is for the treatment of overactive bladder with nocturia.

24. The pharmaceutical composition according to claim 17, wherein the composition is for the treatment of overactive bladder with nocturia in women.

25. The pharmaceutical composition according to claim 1, wherein the second active ingredient is a selective alpha-blocker.

26. The pharmaceutical composition according to claim 25, wherein the selective alpha-blocker is tamsulosin, a prodrug form thereof, or a pharmaceutically acceptable salt thereof.

27. The pharmaceutical composition according to claim 1, wherein the composition is in an oral dosage form.

28. The pharmaceutical composition according to claim 27, wherein the composition is adapted for sublingual administration.

29. (canceled)

30. (canceled)

31. A process for preparing a pharmaceutical composition comprising sublimating a solvent from a liquid preparation comprising a first pharmaceutically active ingredient, one or more matrix-forming agents, controlled released pellets comprising a second pharmaceutically active ingredient, and a solvent.

32. The process according to claim 31, wherein the sublimation is carried out by freeze drying the liquid preparation.

33. The process according to claim 31, wherein the solvent is water.

34. A process for preparation of a pharmaceutical composition comprising the steps of:
(a) preparing a mixture comprising a first active ingredient, controlled release beads comprising a second active ingredient, one or more matrix-forming agents, and a solvent;
(b) freezing the mixture; and
(c) sublimating the solvent from the frozen mixture,
wherein the pharmaceutical composition so obtained disintegrates within 30 seconds upon contact with a standardized aqueous medium.

35. The process according to claim 33, wherein the composition disintegrates within 10 seconds upon contact with a standardized aqueous medium.

36. The process according to claim 31 or 34, wherein the composition comprises an open matrix network comprising a first pharmaceutically active ingredient; one or more matrix-forming agents; and controlled release beads comprising a second pharmaceutically active ingredient.

37. A method for treating overactive bladder, nocturia or a combination thereof in a subject in need thereof, which comprises administering to the subject a therapeutically effective amount of a pharmaceutical composition comprising: an open matrix network comprising a first pharmaceutical active ingredient; one or more matrix-forming agents; and controlled release beads comprising a second pharmaceutical active ingredient of an antimuscarinic compound.

38. The method according to claim 37 wherein the subject is a female subject.

39. A method for treating benign prostatic hyperplasia in a subject in need thereof, which comprises administering to the subject a therapeutically effective amount of a pharmaceutical composition comprising: an open matrix network comprising a first pharmaceutical active ingredient; one or more matrix-forming agents; and controlled release beads comprising a second pharmaceutical active ingredient of a selective alpha-blocker.

40. The method according to claim 39 wherein the subject is a male subject.
